(19) United States
(12) Patent Application Publication
(10) Pub. No.: US 2026/0083398 A1
(43) Pub. Date: Mar. 26, 2026

(54) Systems and Methods for Real-Time Hydration

(71) Applicants: Leonid Shapiro, Englewood, NJ (US); Dimitri Ivanovski, Lyndhurst, NJ (US)

(72) Inventors: Leonid Shapiro, Englewood, NJ (US); Dimitri Ivanovski, Lyndhurst, NJ (US)

(21) Appl. No.: 19/340,387

(22) Filed: Sep. 25, 2025

Related U.S. Application Data
(60) Provisional application No. 63/699,102, filed on Sep. 25, 2024.
(60) Provisional application No. 63/751,156, filed on Jan. 29, 2025.

(51) Int. Cl. A61B 5/00; A61B 5/01; A61B 5/026

(52) U.S. Cl. CPC A61B 5/4875; A61B 5/01; A61B 5/026; A61B 5/4809; A61B 5/4833; A61B 5/7203; A61B 5/7246; A61B 5/725; A61B 5/7267; A61B 5/7275; A61B 2562/0271

(57) ABSTRACT

The present disclosure provides a system for hydration monitoring and alerting. The system includes a server configured to obtain a set of photoplethysmogram (PPG) data samples for a set of population users, mark each PPG data sample with a selected label indicating a hydration level, and generate a model based on the resulting training data. The system also includes an interface-sensor system with a PPG sensor configured to obtain a current PPG data sample for a user and a user interface. The user interface is configured to obtain the current PPG data sample, process it through the model to obtain a hydration score, determine a hydration state based on the score, and output an indicator based on the hydration state.

### CROSS-REFERENCE TO RELATED APPLICATION(S)

This application claims the benefit of U.S. Provisional Application No. 63/699,102, filed Sep. 25, 2024, entitled “System and Methods for Real-time Hydration Monitoring Using PPG and Machine Learning”; and U.S. Provisional Application No. 63/751,156, filed Jan. 29, 2025, entitled “System and Methods for Detecting Maximum Hydration and Dehydration Using PPG and Machine Learning.”

The contents of each of the above referenced applications are hereby incorporated by reference in their entirety.

### TECHNICAL FIELD

Various aspects of the present disclosure relate generally to systems and methods for hydration monitoring and, more particularly, to systems and methods for hydration monitoring and alerting using photoplethysmogram (PPG) data and machine learning techniques.

### BACKGROUND

Hydration is a critical factor in maintaining overall health and wellness. Proper hydration is essential for various bodily functions, including regulating body temperature, transporting nutrients, maintaining blood volume, and supporting organ function. Despite its importance, many individuals struggle to maintain adequate hydration levels throughout their daily activities.

Traditional methods of assessing hydration status often rely on subjective measures such as thirst sensation or urine color, which can be unreliable indicators of true hydration levels. More accurate methods, such as blood tests or urine specific gravity measurements, are typically invasive, time-consuming, or require specialized equipment, making them impractical for continuous monitoring in everyday life.

The advent of wearable technology has opened new possibilities for non-invasive, continuous monitoring of various physiological parameters. Photoplethysmography (PPG) is a simple optical technique widely used in wearable devices to measure blood volume changes in the microvascular bed of tissue. While PPG is commonly used for heart rate monitoring, its potential for assessing hydration status has not been fully explored.

Machine learning techniques have shown promise in extracting meaningful information from complex physiological data. The application of these techniques to PPG data could potentially provide insights into hydration status that were previously difficult to obtain non-invasively and in real-time.

However, interpreting PPG signals for hydration assessment presents several challenges. PPG measurements can be influenced by various factors such as skin properties, ambient temperature, and physical activity. Additionally, the relationship between PPG signals and hydration status is complex and may vary among individuals.

There is a need for improved systems and methods that can accurately and continuously monitor hydration status using non-invasive techniques. Such systems could provide valuable information to individuals, athletes, and healthcare providers, enabling timely interventions to maintain optimal hydration levels and improve overall health outcomes.

The present disclosure is directed to overcoming one or more of these above-referenced challenges.

### SUMMARY OF THE DISCLOSURE

According to certain aspects of the disclosure, systems, methods, and computer readable memory are disclosed for hydration monitoring.

In some cases, a system for hydration monitoring and alerting may include: a server, and an interface-sensor system.

In some cases, the server may be configured to: obtain a set of photoplethysmogram (PPG) data samples for a set of population users; mark each PPG data sample of the set of PPG data samples with a selected label selected from a set of labels, to thereby obtain a set of training data, wherein the selected label indicates a hydration level of a population user of the set of population users at a specific time that corresponds to when a respective PPG data sample was obtained, and the set of labels includes at least two labels that indicate different hydration levels of the set of population users, and the at least two labels include a first label for dehydration and a second label for hydration; and generate a model based on the set of training data.

In some cases, the user-sensor system may include: a PPG sensor configured to obtain a current PPG data sample for a user; and a user interface configured to obtain the current PPG data sample from the PPG sensor, and indicate a hydration state of the user based on the current PPG data sample.

In some cases, the user interface may be configured to: obtain the model; process the current PPG data sample through the model to obtain a hydration score for the user; based on the hydration score of the user, determine a hydration state of the user; and output an indicator based on the hydration state.

Additional objects and advantages of the disclosed technology will be set forth in part in the description that follows, and in part will be apparent from the description, or may be learned by practice of the disclosed technology.

It is to be understood that both the foregoing general description and the following detailed description are exemplary and explanatory only and are not restrictive of the disclosed technology, as claimed.

### BRIEF DESCRIPTION OF THE DRAWINGS

The accompanying drawings, which are incorporated in and constitute a part of this specification, illustrate various exemplary aspects and together with the description, serve to explain the principles of the disclosed technology.

FIG. 1 illustrates a block diagram of a hydration monitoring system, according to aspects of the present disclosure.

FIG. 2 illustrates a system diagram of a hydration monitoring system, according to an embodiment.

FIG. 3 illustrates a system diagram of a server for hydration monitoring and alerting, according to aspects of the present disclosure.

FIG. 4 illustrates a block diagram of a model for processing photoplethysmogram data to determine hydration status, according to an embodiment.

FIG. 5A illustrates a sequence diagram of a connection and setup loop process, according to aspects of the present disclosure.

FIG. 5B illustrates a sequence diagram of a routine loop process for data collection and storage, according to an embodiment.

FIG. 5C illustrates a sequence diagram of a process for training or retraining a model, according to aspects of the present disclosure.

FIG. 5D illustrates a sequence diagram of a prediction loop process, according to an embodiment.

FIG. 6A illustrates a visualization of sample data showing hydration states over time, according to aspects of the present disclosure.

FIG. 6B illustrates a visualization of sample data representing hydration states over time, according to an embodiment.

FIG. 6C illustrates a visualization of predictions using a trained model for hydration monitoring, according to aspects of the present disclosure.

FIG. 6D illustrates a graph of raw photoplethysmogram data over time, according to an embodiment.

FIG. 6E illustrates a spectrogram of a photoplethysmogram signal, according to aspects of the present disclosure.

FIG. 6F illustrates a generated photoplethysmogram signal based on raw data, according to an embodiment.

FIG. 7 illustrates a flowchart for a method of collecting hydration data and training a model, according to aspects of the present disclosure.

FIG. 8 illustrates a flowchart for a method of hydration monitoring and model updating, according to an embodiment.

FIG. 9 illustrates a flowchart of a method for determining a user's maximum dehydration state, according to an embodiment.

FIG. 10 illustrates a flowchart of a method for determining a user's maximum hydration state, according to an embodiment.

FIG. 11 illustrates a model for processing photoplethysmogram data with neural network architecture, according to aspects of the present disclosure.

FIG. 12 illustrates BMI adjustment flowcharts for hydration parameter modifications, according to aspects of the present disclosure.

FIG. 13A illustrates a start user interface sequence for a hydration monitoring application, according to aspects of the present disclosure.

FIG. 13B illustrates a setup user interface sequence for the hydration monitoring application of FIG. 13A, according to aspects of the present disclosure.

FIG. 13C illustrates a snapshot user interface for displaying hydration monitoring data, according to aspects of the present disclosure.

FIG. 13D illustrates an add fluid data user interface sequence for the hydration monitoring application, according to aspects of the present disclosure.

FIG. 13E illustrates an analytics dashboard user interface for displaying hydration insights, according to aspects of the present disclosure.

FIG. 13F illustrates a settings control user interface for the hydration monitoring application, according to an embodiment.

FIG. 14 depicts an example system that may execute techniques presented herein.

### DETAILED DESCRIPTION

The present disclosure provides a system for real-time hydration monitoring and alerting. This system leverages photoplethysmogram (PPG) data and machine learning techniques to assess an individual's hydration status. The system includes a server and an interface-sensor system. The server is configured to obtain a set of PPG data samples for a set of population users and mark each PPG data sample with a selected label indicating a hydration level of a population user at a specific time. The labels include at least two labels that indicate different hydration levels of the population users. The server is also configured to generate a model based on the set of training data.

The interface-sensor system includes a PPG sensor and a user interface. The PPG sensor is configured to obtain a current PPG data sample for a user. The user interface is configured to obtain the current PPG data sample from the PPG sensor, and indicate a hydration state of the user based on the current PPG data sample. The user interface is configured to obtain the model, process the current PPG data sample through the model to obtain a hydration score for the user, determine a hydration state of the user based on the hydration score, and output an indicator based on the hydration state.

In some cases, the system may provide valuable information to individuals, athletes, and healthcare providers, enabling timely interventions to maintain optimal hydration levels and improve overall health outcomes. The system may offer a non-invasive, continuous monitoring solution for hydration status, providing insights into hydration levels that were previously difficult to obtain non-invasively and in real-time.

Thus, methods and systems of the present disclosure may be improvements to computer technology and/or hydration monitoring.

### 1. Hydration Monitoring System

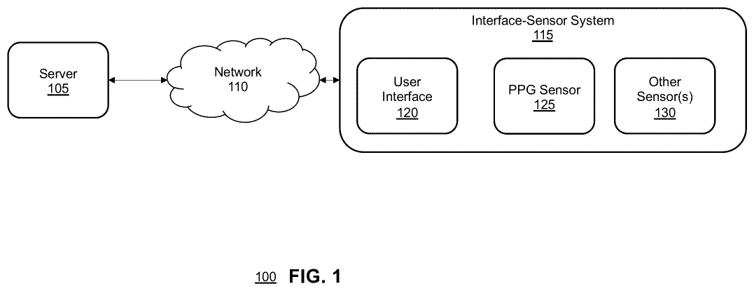

FIG. 1 depicts a hydration monitoring system 100. The hydration monitoring system 100 comprises a server 105 and an interface-sensor system 115. The server 105 is configured to process data and manage communications with the interface-sensor system 115. The interface-sensor system 115 includes a user interface 120 and a photoplethysmogram (PPG) sensor 125.

The PPG sensor 125 is configured to obtain photoplethysmogram data from a user. This data is then transmitted to the user interface 120, which provides a means for the user to interact with the system and receive hydration-related information. The user interface 120 is also responsible for obtaining the PPG data from the PPG sensor 125 and processing it for further analysis.

The server 105 and the interface-sensor system 115 are connected via a network 110, which facilitates communication between the two components. The network 110 can be any type of network capable of transmitting data, such as a local area network (LAN), a wide area network (WAN), or the internet.

In some cases, the server 105 may be configured to process the PPG data obtained from the PPG sensor 125, generate a model based on the processed data, and send the model back to the user interface 120 via the network 110. The user interface 120 can then use the model to determine the user's hydration status and provide feedback to the user.

In some aspects, the server 105 may also be configured to store the PPG data and the generated model for future reference. This allows the system to track the user's hydration status over time and make more accurate predictions about the user's hydration needs.

In some cases, the PPG sensor 125 may be a wearable device, such as a wristband or a watch, that the user can wear throughout the day. The PPG sensor 125 may be configured to continuously monitor the user's PPG data and transmit the data to the user interface 120 in real-time. This allows the hydration monitoring system 100 to provide real-time hydration monitoring and alerting services to the user.

In some aspects, the user interface 120 may be a mobile application installed on a user's smartphone or tablet. The user interface 120 may provide a user-friendly interface for the user to interact with the hydration monitoring system 100, view their hydration status, and receive alerts and recommendations based on their hydration status. In some cases, the user interface 120 may be implemented on various device platforms including extended reality (XR) devices, augmented reality (AR) headsets, virtual reality (VR) systems, smartwatches, fitness trackers, or other wearable computing devices that may provide immersive or convenient access to hydration monitoring information and controls.

In some cases, the hydration monitoring system 100 may also include additional components, such as a battery for powering the PPG sensor 125 and the user interface 120, a memory for storing the PPG data and the generated model, and a processor for processing the PPG data and generating the model.

In some aspects, the hydration monitoring system 100 may be configured to work with multiple users simultaneously. Each user may have their own PPG sensor 125 and user interface 120, and the server 105 may be configured to process the PPG data from each user separately and generate a separate model for each user. This allows the hydration monitoring system 100 to provide personalized hydration monitoring and alerting services to each user.

In some cases, the interface-sensor system 115 may include other sensors 130 in addition to the PPG sensor 125. The other sensors 130 may comprise various types of physiological monitoring devices configured to capture complementary data that enhances hydration assessment accuracy. In some aspects, the other sensors 130 may include a temperature sensor configured to measure body temperature, which may correlate with dehydration states, particularly during physical activity when core temperature rises due to fluid loss through perspiration and respiratory water loss.

In some cases, the other sensors 130 may include additional physiological monitoring devices such as skin conductance sensors, heart rate variability sensors, or ambient environmental sensors. The skin conductance sensors may detect changes in skin moisture and electrical conductivity that may indicate hydration status. Environmental sensors may monitor ambient temperature, humidity, and other conditions that affect hydration needs and physiological responses.

In some aspects, the other sensors 130 may be configured to operate in coordination with the PPG sensor 125 to provide synchronized multi-parameter data collection. The server 105 may be configured to process data from multiple sensor types simultaneously, incorporating temperature measurements, environmental conditions, and other physiological parameters into the model generation process. This multi-modal approach may enhance the accuracy of hydration state determination by leveraging multiple physiological indicators rather than relying solely on PPG data.

The system may incorporate additional physiological monitoring capabilities that provide complementary feedback mechanisms for enhanced hydration assessment accuracy. In some aspects, the other sensors may include temperature monitoring devices that demonstrate convergent trends with dehydration indicators, where progressive dehydration correlates with increasing core body temperature rather than divergent patterns, providing confirmatory physiological evidence of fluid balance changes. The system may be configured to analyze these convergent relationships between PPG-derived hydration indicators and thermal measurements to enhance prediction confidence and reduce false positive assessments.

The system may obtain temperature trend data through various methodological approaches ranging from simple single-point measurements to complex multi-site calibration protocols. In some cases, the system may utilize single-point skin temperature sensors that provide basic thermal monitoring capabilities, while more sophisticated implementations may incorporate multi-site temperature measurement using formulas such as the Ramanathan 4-site approach or Hardy-DuBois 7-site methodology that calculate mean skin temperature from multiple body locations. Advanced implementations may employ personal calibration protocols that correlate skin temperature measurements with core temperature references, or two-node core-skin thermal models that solve heat balance equations based on metabolic heat production, environmental conditions, and physiological parameters. In some aspects, the system may implement empirical rule-based approaches that provide temperature trend estimation without requiring complex calibration procedures, enabling practical deployment in consumer applications while maintaining physiologically relevant thermal monitoring capabilities.

The empirical temperature estimation function may operate by applying established physiological relationships between skin temperature measurements and core body temperature trends during exercise conditions. The system may calculate mean body temperature using weighted combinations of estimated core temperature and measured skin temperature, such as the relationship where mean body temperature equals approximately 0.67 times core temperature plus 0.33 times skin temperature during exercise conditions. The function may invert this relationship to estimate core temperature trends by assuming that changes in skin temperature correlate with proportional changes in core temperature, enabling the system to generate estimated core temperature trend lines that indicate thermal stress progression during dehydration states. In some cases, the empirical function may incorporate activity level adjustments, environmental compensation factors, and individual baseline corrections to enhance trend accuracy while maintaining computational simplicity suitable for real-time monitoring applications. The system may display these temperature trend estimates with appropriate uncertainty bands to communicate the empirical nature of the calculations while providing users with actionable thermal stress indicators that correlate with hydration status changes during endurance activities.

In some cases, the system may be designed to use non-invasive hemoglobin and hematocrit concentration monitoring technologies that could provide additional feedback loop capabilities for hydration state determination. The hemoglobin concentration measurements may serve as physiological indicators where dehydration naturally increases hemoglobin concentration as plasma volume shifts from vascular space to interstitial tissues and organs, while hydration processes dilute hemoglobin concentration through increased plasma volume and circulating blood volume restoration. The model and system architecture may incorporate hemoglobin concentration data as supplementary input parameters alongside PPG and temperature measurements, enabling multi-modal physiological assessment that leverages blood composition changes, cardiovascular responses, and thermal regulation patterns to provide comprehensive hydration monitoring capabilities. These additional sensor modalities may operate through the same data collection and processing pathways as existing sensors, with the model architecture configured to integrate multiple physiological parameters for enhanced hydration state classification accuracy.

In some cases, the other sensors 130 may transmit data to the user interface 120 via the same communication pathways used by the PPG sensor 125. The user interface 120 may be configured to aggregate data from all sensors and transmit the combined dataset to the server 105 for analysis and model training. This integrated sensor approach may provide a more comprehensive physiological profile for each user, enabling more accurate and personalized hydration monitoring.

### 2. Systems for Hydration Monitoring

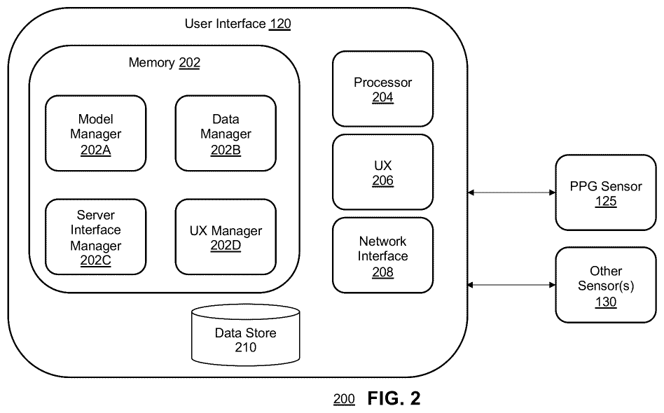

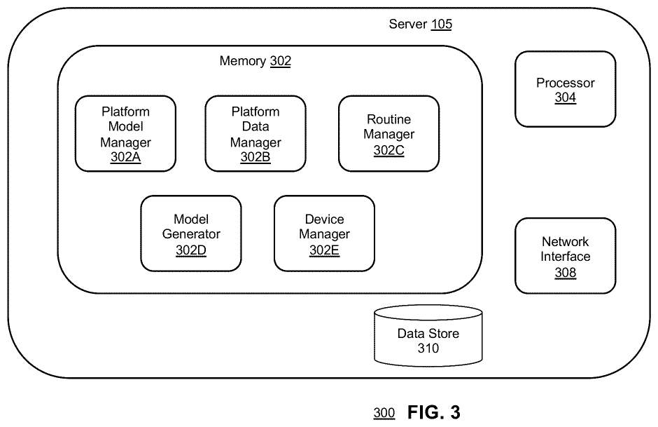

FIGS. 2 and 3 depict system diagrams of the user interface 120 and the server 105, respectively. The features of FIGS. 2 and 3 may apply to any of the features of FIGS. 1, 4, 5A-5D, 6A-6F. 7 and 8.

FIG. 2 depicts a system diagram 200 of the user interface 120. The system diagram 200 includes the user interface 120 and the PPG sensor 125. The user interface 120 comprises several components that work together to process and manage data from the PPG sensor 125.

The user interface 120 contains a memory 202 which houses four management modules: a model manager 202A, a data manager 202B, a server interface manager 202C, and a UX manager 202D. These modules handle various aspects of data processing and system operations. The model manager 202A is responsible for managing the hydration model used to interpret the PPG data. The data manager 202B oversees the storage and retrieval of data within the system. The server interface manager 202C manages the communication between the user interface 120 and the server 105. The UX manager 202D manages the user experience, including the display of hydration status and alerts to the user.

A processor 204 is included in the user interface 120 to execute instructions and perform computations necessary for the operation of the various software modules stored in memory 202. The user interface 120 also features a UX 206, which provides the interface for user interactions with the system. The UX 206 may display the user's current hydration status, provide alerts when the user's hydration level falls below or rises above certain thresholds, and provide recommendations for maintaining optimal hydration.

For external communications, the user interface 120 includes a network interface 208. This allows the system to connect with other devices or networks as needed, such as the server 105 or a user's smartphone.

The user interface 120 also contains a data store 210, which serves as a repository for storing data used by the system. This data may include PPG data, hydration status data, user settings, and other relevant information.

The PPG sensor 125 is shown connected to the user interface 120, indicating that it provides input data to the system. This sensor captures photoplethysmogram data from the user for analysis by the components within the user interface 120. The PPG sensor 125 may be a wearable device, such as a wristband or a watch, that the user can wear throughout the day. The PPG sensor 125 may be configured to continuously monitor the user's PPG data and transmit the data to the user interface 120 in real-time.

In some aspects, the PPG sensor 125 and the user interface 120 may be integrated into a single device, such as a smartwatch or a fitness tracker. In other cases, the PPG sensor 125 and the user interface 120 may be separate devices that communicate wirelessly. For example, the PPG sensor 125 could be a wearable device that transmits PPG data to a smartphone running a mobile application that serves as the user interface 120.

In some aspects, the interface-sensor system may include other sensors 130 that provide additional physiological data alongside the PPG sensor 125. The other sensors 130 may be configured to obtain various types of physiological measurements that complement PPG data for enhanced hydration assessment. In some cases, the other sensors 130 may include a temperature sensor configured to continuously monitor body temperature, providing thermal data (e.g., trend data for core body temperature) that correlates with hydration status and may be particularly valuable during exercise or environmental stress conditions.

In some cases, the data manager 202B may be configured to receive and store data from the other sensors 130 in addition to PPG data. The data manager 202B may synchronize data collection from multiple sensor types, ensuring that temperature measurements, environmental readings, and other sensor data are time-aligned with PPG samples for accurate correlation analysis. This synchronized multi-parameter data collection may enable the model manager 202A to process comprehensive physiological datasets when determining hydration states.

In some aspects, the other sensors 130 may provide input data that is processed by the model manager 202A as additional feature parameters alongside PPG-derived features. The model manager 202A may be configured to integrate temperature data, skin conductance measurements, or other sensor inputs into the hydration assessment algorithm. This multi-modal processing approach may improve the accuracy of hydration state determination by leveraging multiple physiological indicators that respond to changes in hydration status.

In some cases, the server interface manager 202C may be configured to transmit data from the other sensors 130 to the server 105 for inclusion in population-based model training. During routine data collection phases, the other sensors 130 may provide additional training parameters that enhance the server's ability to generate robust models capable of recognizing hydration states across diverse physiological conditions. The UX manager 202D may display information from the other sensors 130 to provide users with comprehensive feedback about their physiological status beyond hydration alone.

In some aspects, the other sensors 130 may operate in different sampling modes corresponding to the PPG sensor's sampled and continuous modes. During sampled mode operation, the other sensors 130 may collect measurements at regular intervals or upon user request. During continuous mode operation, particularly during physical activity, the other sensors 130 may provide high-frequency data collection to capture rapid physiological changes that may indicate evolving hydration status. This adaptive sampling approach may optimize battery life while ensuring adequate data resolution for accurate hydration monitoring.

FIG. 3 depicts a system diagram 300 of the server 105 for hydration monitoring and alerting. The server 105 includes several components that work together to process and manage hydration-related data.

The server 105 comprises a memory 302, which contains multiple software modules. These modules include a platform model manager 302A, a platform data manager 302B, a routine manager 302C, a model generator 302D, and a device manager 302E. Each of these modules performs specific functions related to data processing, model management, and device communication.

The platform model manager 302A is responsible for managing the hydration model used to interpret the PPG data. The platform data manager 302B oversees the storage and retrieval of data within the system. The routine manager 302C manages the execution of routines for data processing and model generation. The model generator 302D is responsible for generating the hydration model based on the training data. The device manager 302E manages the communication between the server 105 and the user interfaces.

A processor 304 is included in the server 105 to execute instructions and perform computations necessary for the operation of the various software modules stored in memory 302. The server 105 also includes a network interface 308, which enables communication with external devices and systems, allowing the server to receive data and transmit results.

A data store 310 is incorporated into the server 105 for storing various types of data, which may include user information, hydration data, and generated models. The data store 310 serves as a repository for storing data used by the system.

In some aspects, the server 105 is configured to obtain a set of photoplethysmogram (PPG) data samples for a set of population users. The server 105 marks each PPG data sample with a selected label from a set of labels to obtain a set of training data. The selected label indicates a hydration level of a population user at a specific time that corresponds to when a respective PPG data sample was obtained. The set of labels includes at least two labels that indicate different hydration levels of the set of population users. The server 105 is also configured to generate a model based on the set of training data. The model is used to interpret the PPG data and determine the hydration status of the user.

### 3. Model

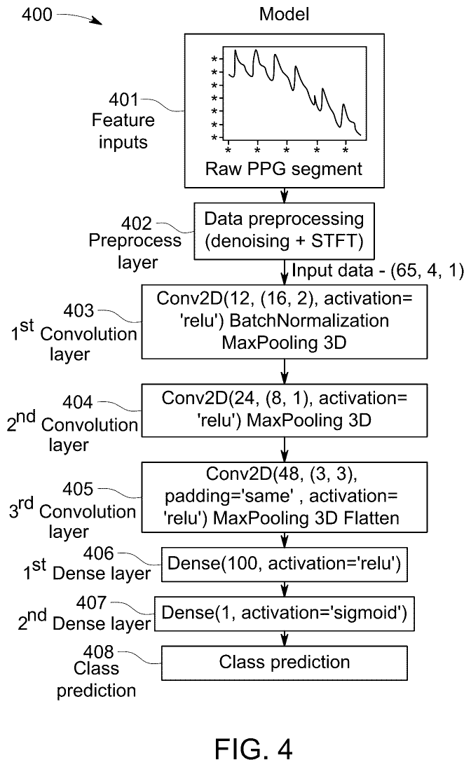

FIG. 4 depicts a block diagram of a machine learning model 400 for processing PPG data to determine hydration status. The features of FIG. 4 may apply to any features of FIGS. 1, 2, 3, 5A-5D, 6A-6F, 7 and 8. The model 400 comprises several components arranged in a sequential processing pipeline.

The model 400 begins with feature inputs 401, which represent the raw PPG segment data. This data is then passed to a preprocess layer 402, which performs data preprocessing operations such as denoising and short-time Fourier transform (STFT). The STFT is a mathematical technique used to transform the time-domain PPG data into the frequency domain. This transformation allows the model to capture both temporal and spectral features of the PPG signal, which can be crucial for accurately determining hydration status.

Following preprocessing, the data flows through a series of convolutional layers. The first convolution layer 403 applies convolution operations with specific parameters, followed by batch normalization and max pooling. The convolution operation in this layer is designed to extract spatial features from the input data. The batch normalization process normalizes the input data for each mini-batch, which can accelerate and stabilize the training process. The max pooling operation reduces data dimensionality by capturing the most significant features, thus mitigating the risk of overfitting.

The second convolution layer 404 and third convolution layer 405 perform similar operations with different parameters, progressively extracting and refining features from the input data. These layers increase the filter count, facilitating the extraction of more intricate features from the PPG data.

After the convolutional layers, the data is processed by two dense layers. The first dense layer 406 and second dense layer 407 apply fully connected neural network operations to further process the extracted features. These layers integrate powerful non-linear combinations of features derived from preceding convolutional layers, aiding the model in learning complex dependencies.

Finally, the output from the dense layers is used to generate a class prediction 408. This prediction represents the model's assessment of the hydration status based on the input PPG data. The class prediction 408 is generated using a sigmoid activation function, which compresses the output to a range of [0, 1], making it ideal for binary classification.

In some aspects, the model 400 may be trained using a set of labeled PPG data samples, where each sample is labeled with a hydration level. The model 400 can then be used to predict the hydration level of a user based on a current PPG data sample. In some cases, the model 400 may be updated or retrained periodically to improve its prediction accuracy.

### 4. Sequence Diagrams for Hydration Monitoring

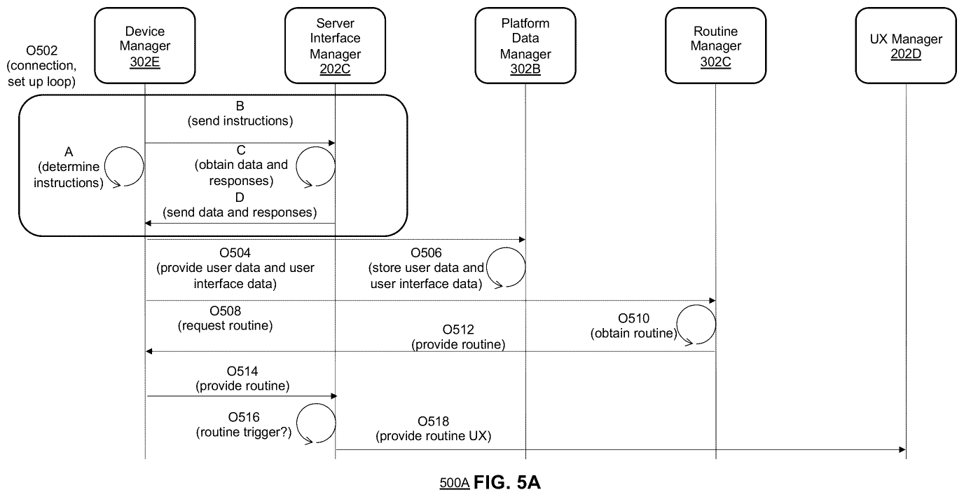

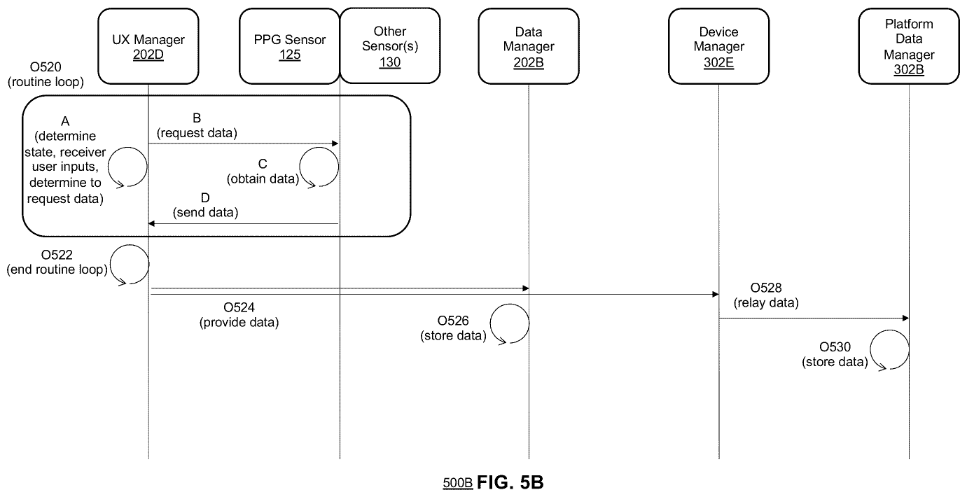

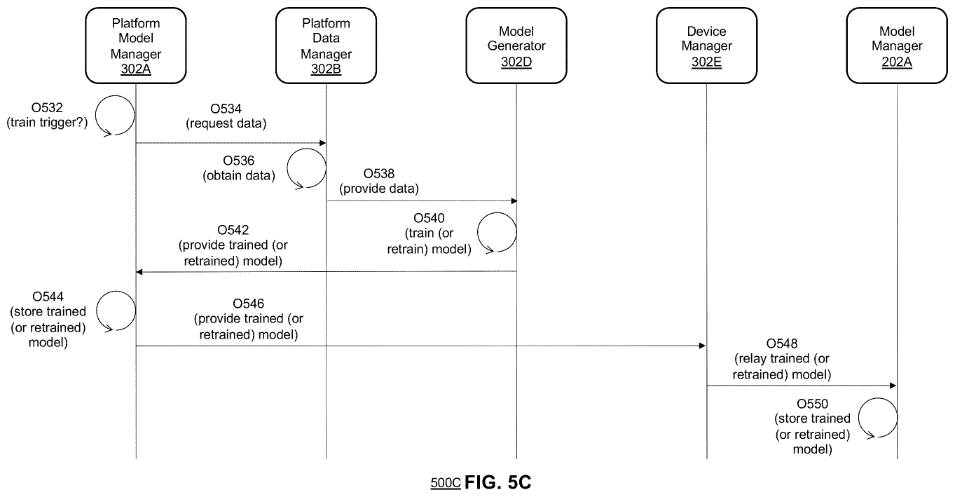

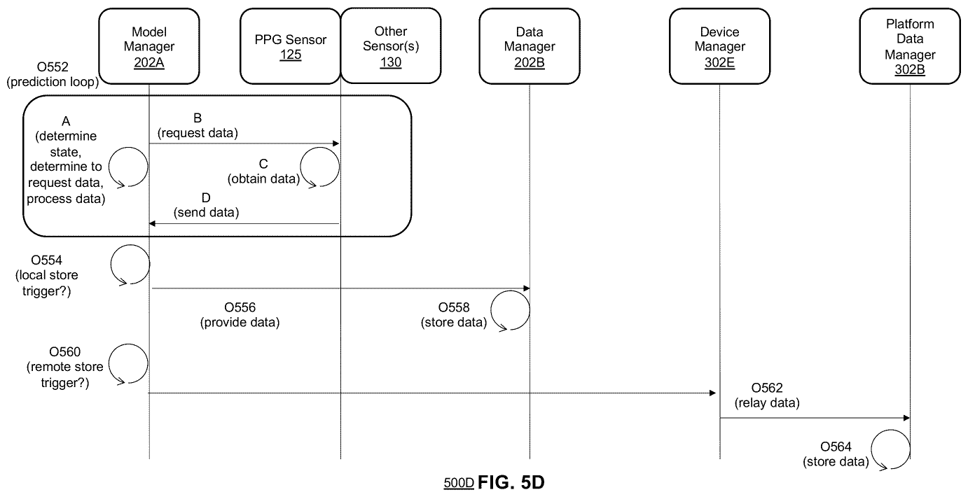

FIGS. 5A, 5B, 5C, and 5D depict sequence diagram 500A, sequence diagram 500B, sequence diagram 500C, and sequence diagram 500D, respectively. The features of FIGS. 5A-5D may apply to any features of FIGS. 1, 2, 3, 4, 6A-6F, 7 and 8.

FIG. 5A depicts a sequence diagram 500A of a connection and setup loop process. The connection and setup loop process involves multiple system components to initialize and prepare the hydration monitoring system for operation.

The process begins with the device manager 302E, which determines instructions in step 0504. These instructions are then sent to the server interface manager 202C in step 0506. The server interface manager 202C obtains data and responses in step 0508, which are subsequently sent back to the device manager in step 0510.

Following this initial exchange, the device manager provides user data and user interface data to the platform data manager 302B in step O504. The platform data manager then stores this user data and user interface data in step O506.

Next, the device manager requests a routine from the routine manager 302C in step O508. The routine manager obtains the routine in step O510 and provides it to the device manager in step O512. The device manager then provides the routine to the server interface manager in step O514.

In step O516, the server Interface manager checks if there is a routine trigger. The server Interface manager provides the routine UX to the UX manager 202D in step O518. This sequence ensures a systematic flow of information and actions between different components of the system during the connection and setup process.

In some aspects, the connection and setup loop process may be performed periodically to ensure that the hydration monitoring system is always ready for operation. In other cases, the connection and setup loop process may be triggered by specific events, such as a user starting a new hydration monitoring session or a change in the user's hydration status.

In some cases, the instructions determined by the device manager 302E in step 0504 may include instructions for configuring the PPG sensor, initializing the hydration model, or setting up the user interface. The data and responses obtained by the server interface manager 202C in step 0508 may include PPG data, hydration status data, user settings, or other relevant information.

In some aspects, the routine requested by the device manager 302E in step O508 may be a data processing routine, a model training routine, or a hydration status prediction routine. The routine UX provided by the server interface manager to the UX manager 202D in step O518 may include user interface updates, alerts, or recommendations based on the user's hydration status.

FIG. 5B depicts a sequence diagram 500B of a routine loop process for data collection and storage. The routine loop process involves multiple system components to gather, transmit, and store PPG data from the sensor to local and platform storage.

The process begins with the UX manager 202D initiating a routine loop O520. Within this loop, the UX manager performs step A, which involves determining the state, receiving user inputs, and/or deciding to request data. Following this, in step B, the UX manager sends a request for data to the PPG sensor 125. The PPG sensor then executes step C to obtain the requested data. Once the data is obtained, the PPG sensor sends the data back to the UX manager in step D. After receiving the data, the UX manager determine whether to end the routine loop in step O522.

In some aspects, the routine loop process may incorporate data collection from the other sensors 130 alongside the PPG sensor 125. During the routine loop O520, the UX manager 202D may coordinate simultaneous data requests to both the PPG sensor 125 and the other sensors 130, ensuring synchronized multi-parameter data collection. The other sensors 130 may provide temperature measurements, skin conductance readings, or environmental data that complement the PPG data for comprehensive hydration assessment. This coordinated data collection approach may enable the system to capture correlations between different physiological parameters and hydration status, enhancing the quality of training data transmitted to the server 105 for model development.

The UX manager then provides the collected data to both the data manager 202B and the device manager 302E in step O524. Upon receiving the data, the data manager performs step O526 to store the data locally. Simultaneously, the device manager relays the data to the platform data manager 302B in step O528. Finally, the platform data manager executes step O530 to store the received data, completing the data collection and storage process.

In some aspects, the routine loop process may be performed periodically to ensure that the hydration monitoring system is always ready for operation. In other cases, the routine loop process may be triggered by specific events, such as a user starting a new hydration monitoring session or a change in the user's hydration status.

In some cases, the instructions determined by the UX manager 202D in step A may include instructions for configuring the PPG sensor, initializing the hydration model, or setting up the user interface. The data and responses obtained by the PPG sensor 125 in step C may include PPG data, hydration status data, user settings, or other relevant information.

In some aspects, the routine requested by the UX manager 202D in step B may be a data processing routine, a model training routine, or a hydration status prediction routine. The routine UX provided by the PPG sensor 125 to the UX manager 202D in step D may include user interface updates, alerts, or recommendations based on the user's hydration status.

FIG. 5C depicts a sequence diagram 500C of the process of training or retraining a model in a distributed system. The process begins with the platform model manager 302A initiating a train trigger in step O532. This triggers a request for data sent to the platform data manager 302B in step O534. The platform data manager then obtains the necessary data in step O536 and provides it to the model generator 302D in step O538. Upon receiving the data, the model generator proceeds to train or retrain the model in step O540. Once the training or retraining is complete, the model generator provides the trained or retrained model back to the platform model manager in step O542. The platform model manager then stores the newly trained or retrained model in step O544. Following this, the platform model manager provides the trained or retrained model to the device manager 302E in step O546. The device manager then relays the trained or retrained model to the model manager 202A in step O548. Finally, the model manager stores the received trained or retrained model in step O550, completing the sequence of model training and distribution.

In some aspects, the model may be trained using a set of labeled PPG data samples, where each sample is labeled with a hydration level. The model can then be used to predict the hydration level of a user based on a current PPG data sample. In some cases, the model may be updated or retrained periodically to improve its prediction accuracy. The trained or retrained model is then distributed to the user interface for use in real-time hydration monitoring.

In some cases, the platform model manager 302A may initiate the train trigger in response to a variety of triggers, such as a predetermined schedule, a request from a user, or a change in the user's hydration status. The platform data manager 302B may obtain the necessary data from a variety of sources, such as the PPG sensor, the user interface, or an external database. The model generator 302D may use a variety of machine learning algorithms to train or retrain the model, depending on the specific requirements of the hydration monitoring system.

In some aspects, the device manager 302E may receive the trained or retrained model from the platform model manager 302A and relay it to the model manager 202A. The model manager 202A may then store the received model for future use. In some cases, the model manager 202A may also provide the model to the user interface for use in real-time hydration monitoring.

FIG. 5D depicts a sequence diagram 500D of a prediction loop process. The prediction loop process involves multiple system components to obtain current PPG data, process it through the model, and determine the user's hydration state.

The process begins with the model manager 202A at step 0552, which performs a prediction loop process. The prediction loop process starts at step A to determine the state, determine to request data, process data, and processes received data. These instructions are then sent to the PPG sensor 125 in step B. The PPG sensor 125 executes step C to obtain the requested data. Once the data is obtained, the PPG sensor 125 sends the data back to the model manager 202A in step D.

The model manager 202A may process the obtained data through the trained model to generate a hydration prediction for the user. The prediction may be based on analysis of the PPG data characteristics, including signal amplitude, frequency components, and temporal patterns that correlate with hydration states. In some aspects, the model manager 202A may apply preprocessing operations to the obtained data before generating the prediction, such as noise filtering or feature extraction. The resulting hydration prediction may be used to determine the user's current hydration state and trigger appropriate alerts or recommendations through the user interface.

In some cases, the prediction loop process may utilize data from the other sensors 130 in addition to PPG data for enhanced hydration state determination. During step 0554, the model manager 202A may determine to request data from both the PPG sensor 125 and the other sensors 130, processing the combined multi-parameter dataset through the trained model. The other sensors 130 may provide real-time temperature, environmental, or additional physiological measurements that serve as supplementary input features for the neural network predictions. This multi-modal data processing approach may improve the accuracy of hydration score calculation and hydration state determination by leveraging multiple physiological indicators that respond to changes in hydration levels.

After receiving the data, the model manager 202A checks if there is a local store trigger in step 0554. If triggered, the model manager 202A provides the collected data to the data manager 202B in step 0556, which then stores the data locally in step O558.

Simultaneously or separately, if a remote store trigger is activated step 0560, the model manager 202A relays the data to the device manager 302E in step 0562. The device manager 302E then passes it to the platform data manager 302B in step 0564. Finally, the platform data manager 302B executes step 0564 to store the received data, completing the data collection and storage process.

In some aspects, the prediction loop process may be performed periodically to ensure that the hydration monitoring system is always ready for operation. In other cases, the prediction loop process may be triggered by specific events, such as a user starting a new hydration monitoring session or a change in the user's hydration status.

In some cases, the instructions determined by the model manager 202A in step 0554 may include instructions for configuring the PPG sensor, initializing the hydration model, or setting up the user interface. The data and responses obtained by the PPG sensor 125 in step 0558 may include PPG data, hydration status data, user settings, or other relevant information.

### 5. Visualizations

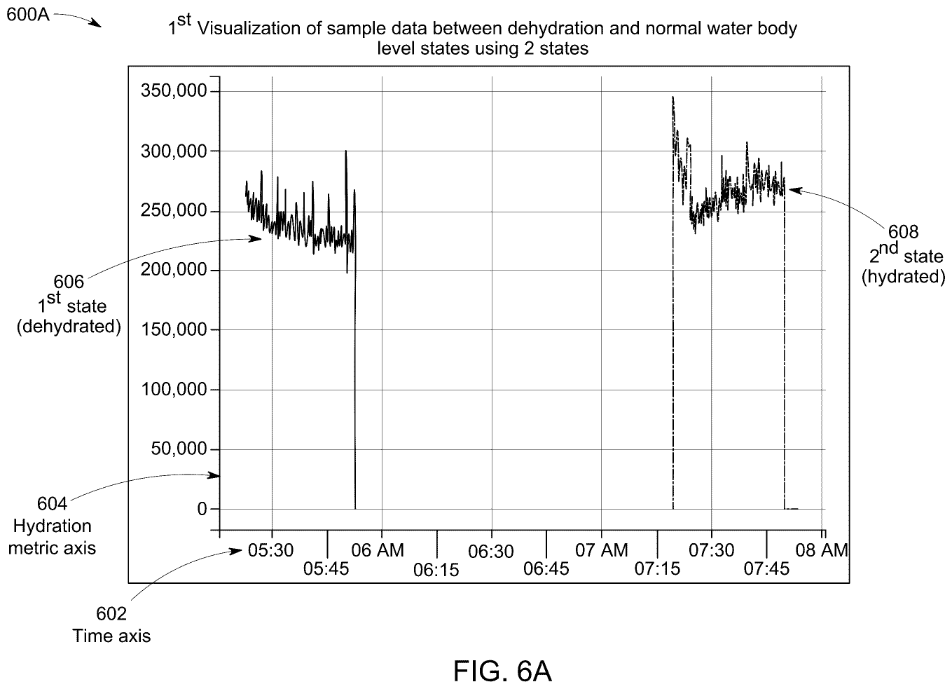

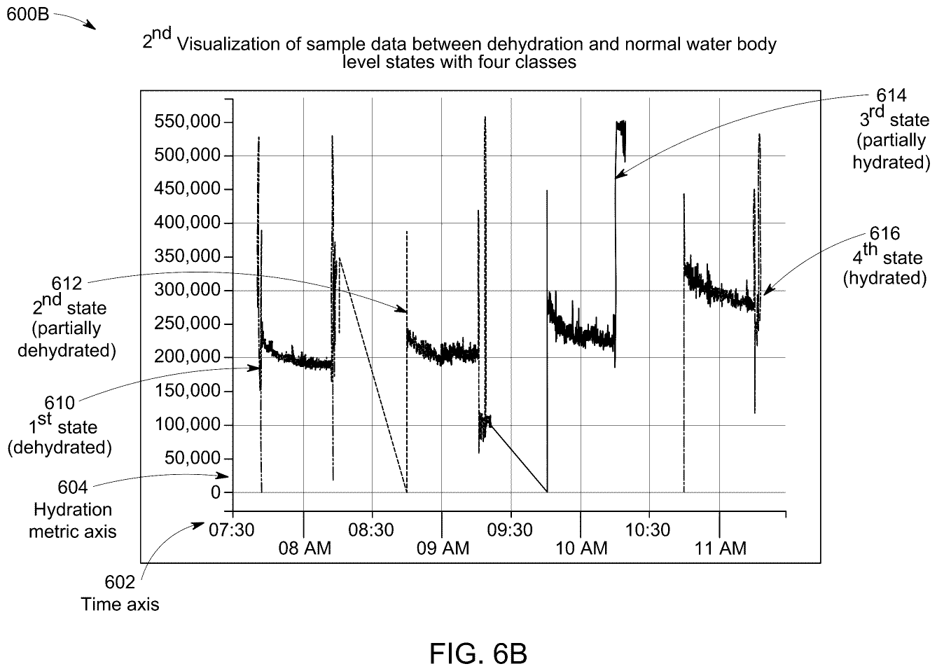

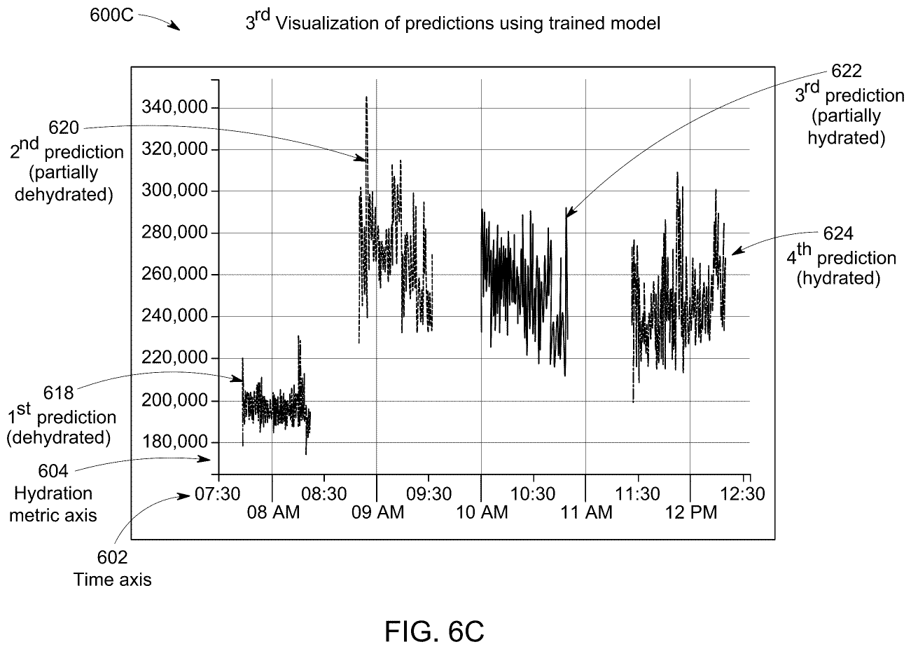

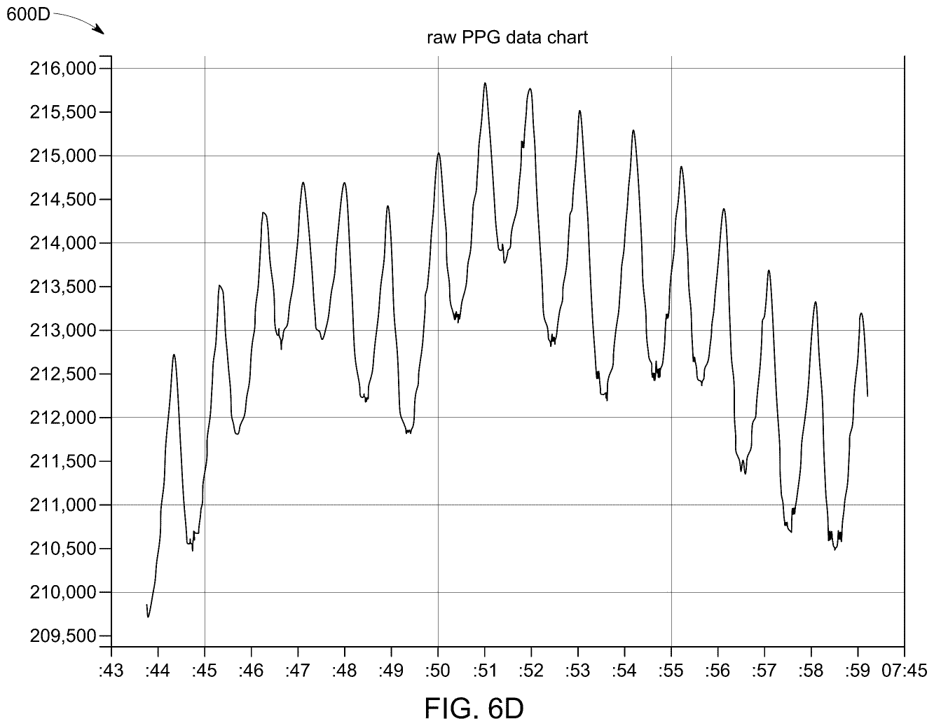

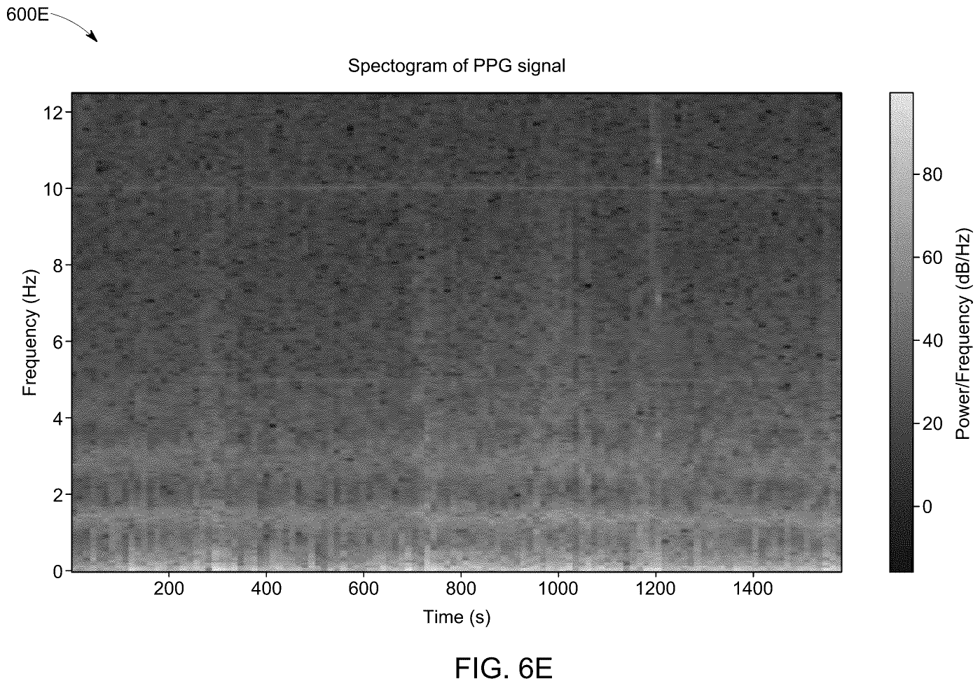

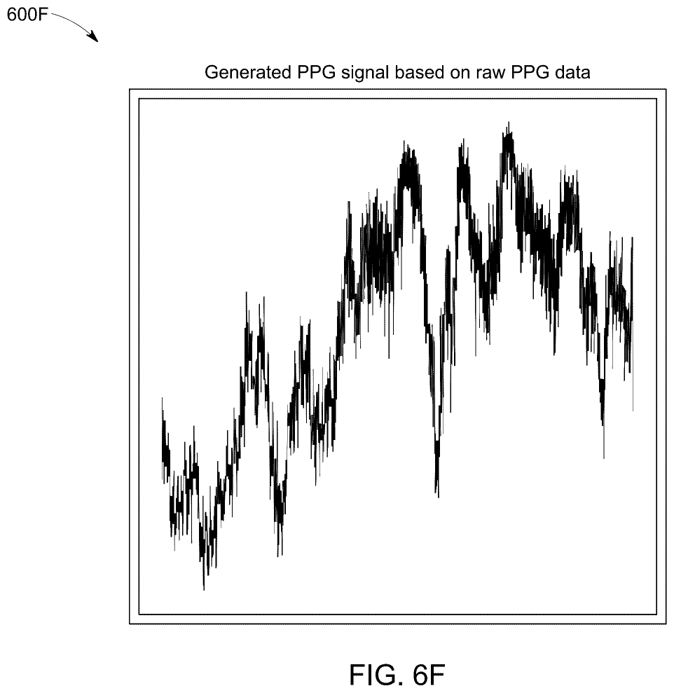

FIGS. 6A-6F depict various features of data, as disclosed herein. The features of FIG. 6A-6F may apply to any features of FIGS. 1, 2, 3, 4, 5A-5D, 7 and 8.

FIG. 6A depicts a visualization 600A of sample data showing hydration states over time. The visualization 600A includes a time axis 602 along the horizontal axis and a hydration metric axis 604 along the vertical axis. Two distinct hydration states are represented: a first state 606, indicating a dehydrated state, and a second state 608, indicating a hydrated state.

The first state 606 occupies the left portion of time on the graph, while the second state 608 is displayed on the right portion of time on the graph. The transition between the first state 606 and the second state 608 is abrupt, occurring around 7:00 AM as indicated on the time axis 602. The hydration metric values for the second state 608 are generally higher than those for the first state 606, as shown on the hydration metric axis 604.

In some aspects, the first state 606 may represent a user's hydration level upon waking up in the morning, after a period of sleep during which the user has not consumed any fluids. The second state 608, on the other hand, may represent the user's hydration level after consuming a certain amount of fluid, such as a liter of water.

The abrupt transition between the first state 606 and the second state 608 may correspond to the user's consumption of fluid. As the user consumes fluid, their hydration level increases, resulting in a shift from the first state 606 to the second state 608.

In some cases, the hydration monitoring system 100 may use the PPG data corresponding to these two states to train the model. Each PPG data sample obtained during the first state 606 may be marked with a first label indicating dehydration, while each PPG data sample obtained during the second state 608 may be marked with a second label indicating hydration.

In this way, the hydration monitoring system 100 can use the labeled PPG data samples to train the model to recognize different hydration states based on PPG data. Once trained, the model can then be used to predict a user's hydration state based on a current PPG data sample, providing real-time hydration monitoring and alerting.

FIG. 6B depicts a visualization 600B of sample data representing multiple hydration states over time. The visualization 600B includes a time axis 602 along the horizontal axis and a hydration metric axis 604 along the vertical axis. The data is represented by four distinct states: a first state 610, representing a dehydrated state; a second state 612, representing a partially dehydrated state; a third state 614, representing a partially hydrated state; and a fourth state 616, representing a hydrated state. Each state is plotted as a series of data points that fluctuate over time, with the first state 610 generally showing lower values on the hydration metric axis 604, and the fourth state 616 showing higher values.

In some aspects, the hydration monitoring system 100 may use four hydration classes for classification. These classes may correspond to different hydration states, such as the dehydrated state represented by the first state 610, the partially dehydrated state represented by the second state 612, the partially hydrated state represented by the third state 614, and the fully hydrated state represented by the fourth state 616. An additional fifth class may represent an over-hydrated state, which is not shown in the visualization 600B.

In some cases, the hydration monitoring system 100 may use the PPG data corresponding to these four states to train the model. Each PPG data sample obtained during each state may be marked with a label indicating the corresponding hydration level. For example, PPG data samples obtained during the first state 610 may be marked with a first label indicating dehydration, PPG data samples obtained during the second state 612 may be marked with a second label indicating partial dehydration, and so on.

In this way, the hydration monitoring system 100 can use the labeled PPG data samples to train the model to recognize different hydration states based on PPG data. Once trained, the model can then be used to predict a user's hydration state based on a current PPG data sample, providing real-time hydration monitoring and alerting.

FIG. 6C depicts a visualization 600C of predictions using a trained model for hydration monitoring. The visualization 600C includes a time axis 602 along the horizontal axis and a hydration metric axis 604 along the vertical axis. The graph shows four distinct predictions over time: a first prediction 618, a second prediction 620, a third prediction 622, and a fourth prediction 624. Each prediction represents a different hydration state, with the first prediction 618 indicating a dehydrated state, the second prediction 620 indicating a partially dehydrated state, the third prediction 622 indicating a partially hydrated state, and the fourth prediction 624 indicating a hydrated state.

In some aspects, the user interface, such as the user interface 120, may determine a hydration state of the user based on the hydration score. The hydration score may be obtained by processing the current PPG data sample through the model. The model, which may be the model 400, is trained to interpret PPG data and determine hydration states. The model may use a variety of machine learning techniques to analyze the PPG data and generate the hydration score. The hydration score may be a numerical value that represents the user's hydration level, with higher scores indicating higher hydration levels.

In some cases, the user interface may use the hydration score to determine the user's hydration state. The hydration state may be one of several predefined states, such as the dehydrated state, the partially dehydrated state, the partially hydrated state, and the fully hydrated state. The user interface may determine the user's hydration state by comparing the hydration score to a set of thresholds associated with the predefined states. For example, if the hydration score falls within a range associated with the partially hydrated state, the user interface may determine that the user is in the partially hydrated state.

In some aspects, the user interface may output an indicator based on the determined hydration state. The indicator may be a visual, auditory, or tactile signal that informs the user of their hydration state. For example, the indicator may be a color-coded bar on the user interface 120, with different colors representing different hydration states. The indicator may also include a recommendation for the user to consume a certain amount of fluid to reach a desired hydration state.

FIG. 6D depicts a graph of raw photoplethysmogram (PPG) data over time. The graph represents a time-varying waveform with fluctuations in amplitude over time. The x-axis represents time in minutes and seconds, ranging from 7:43 to 07:45. The y-axis represents the PPG signal amplitude, ranging from 209,500 to 216,000 in units. The graph shows a red line that fluctuates rapidly over time, with peaks and troughs occurring at regular intervals. The amplitude of the signal generally increases from left to right, with the highest peaks occurring around the middle of the time range. The graph is divided into sections by vertical grid lines at regular intervals.

In some aspects, the raw PPG data represented in the graph may be obtained from a user by the PPG sensor 125. The PPG sensor 125 may be configured to continuously monitor the user's PPG data and transmit the data to the user interface 120 in real-time. This allows the hydration monitoring system 100 to provide real-time hydration monitoring and alerting services to the user.

In some cases, the fluctuations in the PPG signal amplitude over time may correspond to changes in the user's hydration level. For example, a decrease in the amplitude of the PPG signal may indicate a decrease in the user's hydration level, while an increase in the amplitude may indicate an increase in the user's hydration level. The hydration monitoring system 100 may use this information to determine the user's hydration state and provide alerts or recommendations accordingly.

In some aspects, the raw PPG data may be processed by the model manager 202A to generate a hydration score for the user. The hydration score may be a numerical value that represents the user's hydration level, with higher scores indicating higher hydration levels. The model manager 202A may use a variety of machine learning techniques to analyze the PPG data and generate the hydration score.

In some cases, the raw PPG data may be stored in the data store 210 for future reference. This allows the hydration monitoring system 100 to track the user's hydration status over time and make more accurate predictions about the user's hydration needs. The stored PPG data may also be used to train or retrain the model, improving its prediction accuracy.

FIG. 6E depicts a visualization of a spectrogram of a photoplethysmogram (PPG) signal. The spectrogram is a graphical representation of the spectrum of frequencies of the PPG signal as they vary with time. The x-axis of the spectrogram represents time in seconds, ranging from 0 to approximately 1400 seconds. The y-axis represents frequency in Hertz, ranging from 0 to 12 Hz. The scale on the right side of the image indicates the power/frequency intensity.

In some aspects, the spectrogram shows a concentration of higher intensity signals in the lower frequency range, particularly below 2 Hz, with varying patterns and intensities across the time domain. This concentration of higher intensity signals in the lower frequency range may correspond to the heart rate of the user, which is typically in the range of 0.5 to 3 Hz for a resting adult. The varying patterns and intensities across the time domain may reflect changes in the user's hydration status over time.

In some cases, the hydration monitoring system 100 may use the spectrogram of the PPG signal to analyze the user's hydration status. The system 100 may process the PPG signal through a short-time Fourier transform (STFT) to generate the spectrogram. The STFT is a mathematical technique used to transform the time-domain PPG data into the frequency domain. This transformation allows the system 100 to capture both temporal and spectral features of the PPG signal, which can be crucial for accurately determining hydration status.

In some aspects, the system 100 may use machine learning techniques to analyze the spectrogram and determine the user's hydration status. The system 100 may train a model using a set of labeled spectrograms, where each spectrogram is labeled with a hydration level. The system 100 can then use the trained model to predict the hydration level of a user based on a current spectrogram. This approach allows the system 100 to provide real-time hydration monitoring and alerting services to the user based on the analysis of the spectrogram of the PPG signal.

FIG. 6F depicts a graph of a generated photoplethysmogram (PPG) signal based on raw PPG data. The graph represents a time-varying waveform with fluctuations in amplitude over time. The x-axis represents time in minutes and seconds, ranging from 7:43 to 07:45. The y-axis represents the PPG signal amplitude, ranging from 209,500 to 216,000 in units. The graph shows a red line that fluctuates rapidly over time, with peaks and troughs occurring at regular intervals. The amplitude of the signal generally increases from left to right, with the highest peaks occurring around the middle of the time range. The graph is divided into sections by vertical grid lines at regular intervals.

In some aspects, the generated PPG signal may be obtained from a user by the PPG sensor 125. The PPG sensor 125 may be configured to continuously monitor the user's PPG data and transmit the data to the user interface 120 in real-time. This allows the hydration monitoring system 100 to provide real-time hydration monitoring and alerting services to the user.

In some cases, the fluctuations in the PPG signal amplitude over time may correspond to changes in the user's hydration level. For example, a decrease in the amplitude of the PPG signal may indicate a decrease in the user's hydration level, while an increase in the amplitude may indicate an increase in the user's hydration level. The hydration monitoring system 100 may use this information to determine the user's hydration state and provide alerts or recommendations accordingly.

In some aspects, the generated PPG signal may be processed by the model manager 202A to generate a hydration score for the user. The hydration score may be a numerical value that represents the user's hydration level, with higher scores indicating higher hydration levels. The model manager 202A may use a variety of machine learning techniques to analyze the PPG data and generate the hydration score.

In some cases, the generated PPG signal may be stored in the data store 210 for future reference. This allows the hydration monitoring system 100 to track the user's hydration status over time and make more accurate predictions about the user's hydration needs. The stored PPG data may also be used to train or retrain the model, improving its prediction accuracy.

In some aspects, the interface-sensor system 115 may include a PPG sensor 125 that operates using green light with a wavelength range of 525 to 540 nm. This specific wavelength range may be selected due to its efficiency in data handling at the hemoglobin level and tracking of body activity, which can be crucial for accurately determining hydration status. The PPG sensor 125 may be configured to continuously monitor the user's PPG data and transmit the data to the user interface 120 in real-time, thereby enabling the hydration monitoring system 100 to provide real-time hydration monitoring and alerting services to the user.

In some cases, the interface-sensor system 115 may operate in two different modes for data collection: a sampled mode and a continuous mode. In the sampled mode, the PPG sensor 125 may measure the heart rate at regular intervals, such as every five minutes, or whenever a button on the sensor is pressed. This mode may be particularly suitable for resting periods, when the user's hydration status is not expected to change significantly.

In the continuous mode, on the other hand, the PPG sensor 125 may sample the PPG data constantly at a rate of 100 Hz, averaging over four samples. The heart rate may be estimated once per second, providing a more detailed and continuous monitoring of the user's hydration status. This mode may be particularly suitable for active periods, such as during exercise or other physical activities, when the user's hydration status may change rapidly.

In some aspects, the user interface 120 may be configured to switch between the sampled mode and the continuous mode based on the user's activity level or other factors. For example, the user interface 120 may automatically switch to the continuous mode when it detects an increase in the user's heart rate or movement, indicating that the user is engaging in physical activity. Conversely, the user interface 120 may switch back to the sampled mode when it detects that the user's heart rate or movement has returned to normal levels, indicating that the user is at rest.

In some cases, the user interface 120 may also allow the user to manually switch between the sampled mode and the continuous mode. This feature may provide the user with greater control over the hydration monitoring process, allowing them to choose the most appropriate mode based on their current activity level and hydration needs.

### 6. Flowcharts

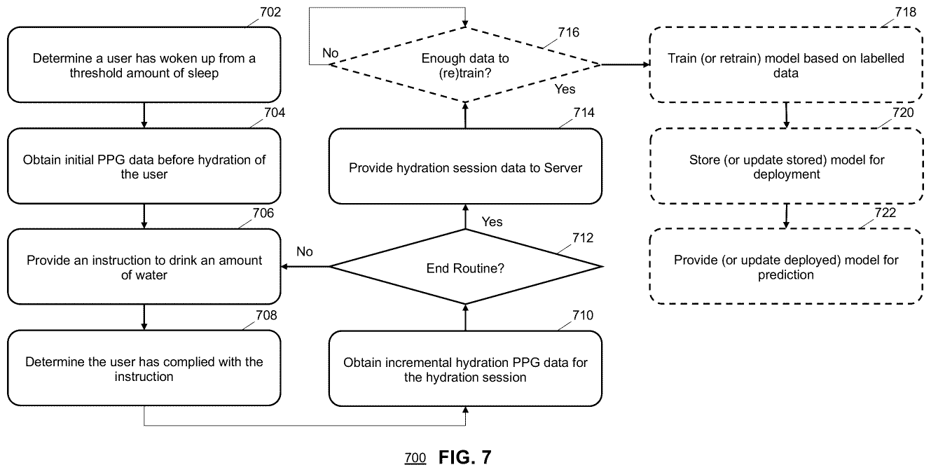

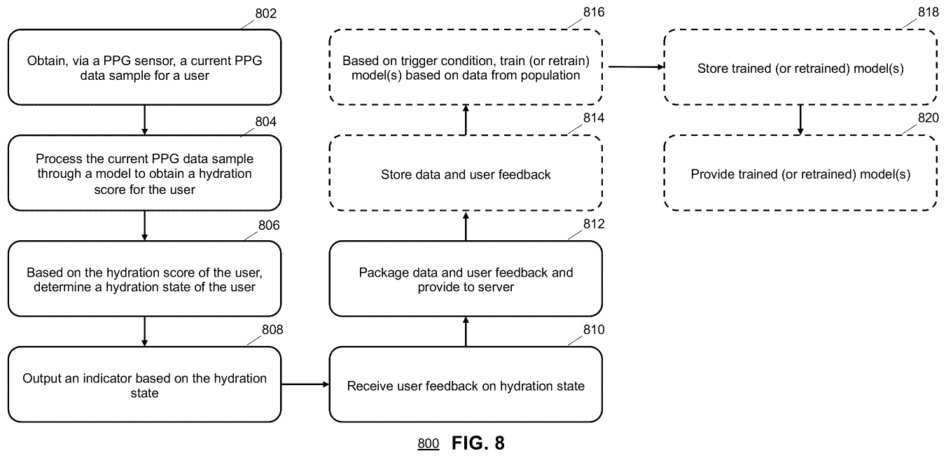

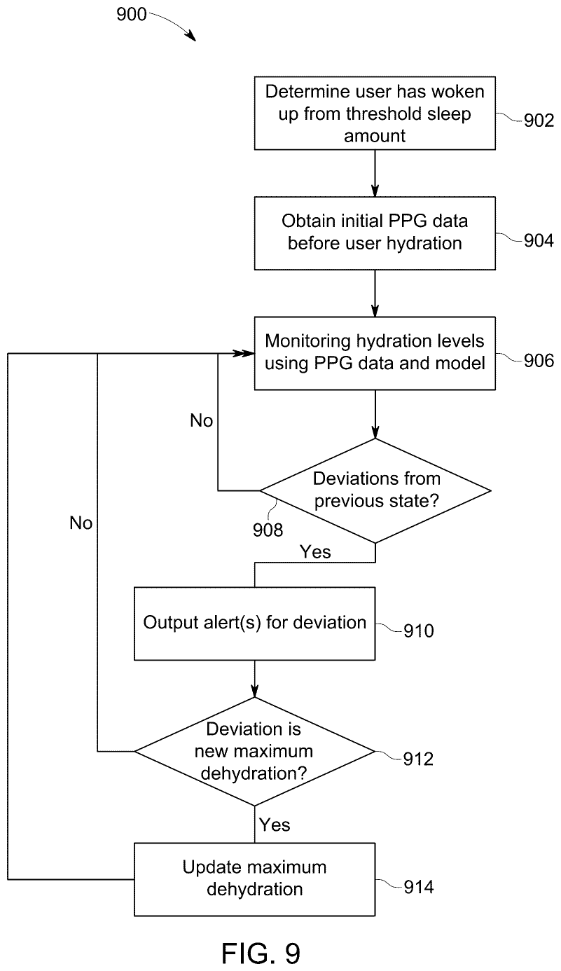

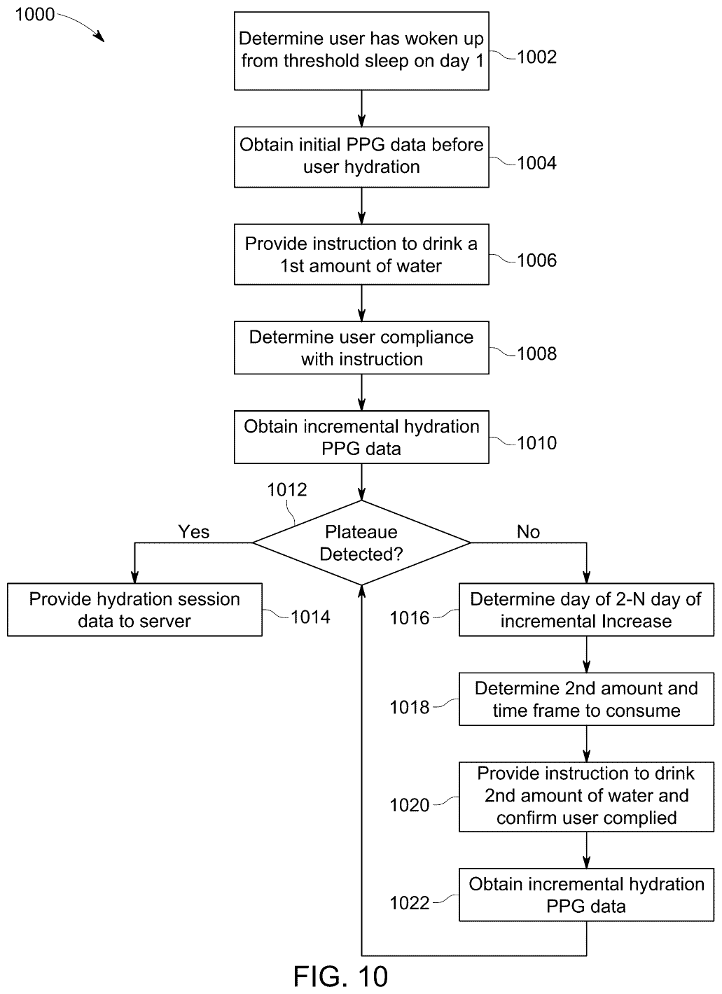

FIGS. 7 and 8 depict flowcharts for hydration monitoring. The features of FIGS. 7 and 8 may apply to any features of FIGS. 1, 2, 3, 4, 5A-5D, and 6A-6F.

The present disclosure provides a system and methods for real-time hydration monitoring and alerting. The system leverages photoplethysmogram (PPG) data and machine learning techniques to assess an individual's hydration status. The system includes a server and an interface-sensor system. The server is configured to obtain a set of PPG data samples for a set of population users and mark each PPG data sample with a selected label indicating a hydration level of a population user at a specific time. The labels include at least two labels that indicate different hydration levels of the population users. The server is also configured to generate a model based on the set of training data.

The interface-sensor system includes a PPG sensor and a user interface. The PPG sensor is configured to obtain a current PPG data sample for a user. The user interface is configured to obtain the current PPG data sample from the PPG sensor, and indicate a hydration state of the user based on the current PPG data sample. The user interface is configured to obtain the model, process the current PPG data sample through the model to obtain a hydration score for the user, determine a hydration state of the user based on the hydration score, and output an indicator based on the hydration state.

In some embodiments, the system may provide valuable information to individuals, athletes, and healthcare providers, enabling timely interventions to maintain optimal hydration levels and improve overall health outcomes. The system may offer a non-invasive, continuous monitoring solution for hydration status, providing insights into hydration levels that were previously difficult to obtain non-invasively and in real-time. Thus, methods and systems of the present disclosure may be improvements to computer technology and/or hydration monitoring.

FIG. 7 depicts a flowchart for a method 700 of collecting hydration data and training a model for hydration prediction. The method 700 begins with step 702, where it is determined that a user has woken up from a threshold amount of sleep. This determination may be made based on various factors, such as the user's sleep schedule, activity level, or other indicators of wakefulness. In some cases, the threshold amount of sleep may be a predetermined duration, such as eight hours, which is generally considered a full night's sleep for most adults.

Following the determination of wakefulness, the method 700 proceeds to step 704, where initial photoplethysmogram (PPG) data is obtained before the user has consumed any fluids. The PPG data may be obtained using a PPG sensor, such as the PPG sensor 125 described herein. The PPG sensor may be configured to continuously monitor the user's PPG data and transmit the data to the user interface in real-time. The initial PPG data obtained at this stage may serve as a baseline for assessing the user's hydration status.

Next, in step 706, an instruction is provided to the user to drink a certain amount of water. The amount of water may be determined based on various factors, such as the user's body weight, activity level, or other individual characteristics. In some cases, the instruction may specify a standard amount of water, such as one liter, which is generally expected to hydrate an average adult an expected amount.

In some aspects, the instruction provided to the user to drink a certain amount of water may be based on a non-linear hydration scale that reflects physiological realities of fluid loss and replacement patterns. The system may implement a population-based training scale utilizing specific fluid volume increments of 0 ml, 500 ml, 700 ml, 800 ml, 900 ml, and 1000 ml. This non-linear progression may recognize that dehydration occurs in predictable patterns, with smaller fluid losses being more common than extreme dehydration states. The initial 500 ml increment may represent a baseline fluid deficit that most individuals experience without conscious awareness, such as overnight fluid loss or mild dehydration from daily activities.

In some cases, the system may provide subsequent instructions following a refined increment pattern, with a 200 ml increment from 500 ml to 700 ml, followed by 100 ml increments for 700 ml to 800 ml, 800 ml to 900 ml, and 900 ml to 1000 ml. This design may acknowledge that moderate dehydration states occur more frequently than severe dehydration, requiring more precise measurement and classification capabilities. For individual-based training, particularly targeting athletes and individuals engaged in endurance activities, the scale may extend beyond the 1-liter population maximum with additional increments of 200-250 ml, potentially reaching 1.2 L, 1.4 L, 1.5 L, or higher depending on individual physiological requirements.

The system may implement different fluid intake scales depending on the target user population and training methodology. For consumer-based applications utilizing incremental daily progression, the system may employ a linear scale with fluid volume increments of 250 ml, 500 ml, 750 ml, 1000 ml, 1250 ml, and 1500 ml over successive training days. This consumer scale may provide consistent 250 ml increments that align with typical daily hydration goals and consumer expectations for gradual fluid intake increases. Alternatively, for athletic or individual data-based training applications, the system may implement a non-linear hydration scale with fluid volume increments of 0 ml, 500 ml, 700 ml, 800 ml, 900 ml, and 1000 ml that reflects physiological realities of fluid loss and replacement patterns. The non-linear athletic scale may provide finer resolution at higher dehydration levels through smaller increments between 700 ml and 1000 ml, recognizing that moderate dehydration states occur more frequently than extreme dehydration and require more precise measurement capabilities. In some cases, the system may support both scaling approaches simultaneously, allowing users to select between consumer-oriented linear progression and athletic-oriented non-linear progression based on their individual hydration monitoring requirements and physiological characteristics. The system may also enable transition between scales, where users may begin with the consumer scale for initial hydration assessment and progress to the athletic scale for enhanced precision in specialized applications such as endurance training or extreme environmental conditions.

The system may accommodate various alternative fluid intake scales with adjustable parameters to optimize hydration monitoring for different applications and user populations. The specific fluid volume values within each scale may be modified based on physiological research, user feedback, or application requirements, allowing the consumer scale increments to be adjusted from the standard 250 ml intervals to alternative values such as 200 ml, 300 ml, or 350 ml depending on target hydration goals. The athletic scale may similarly accommodate different volume specifications, with the initial 500 ml baseline potentially adjusted to 400 ml, 600 ml, or other values that better reflect individual dehydration patterns. The spacing between increments may be implemented as consistent intervals throughout the scale or as variable progressions that provide finer resolution at specific hydration ranges. In some cases, the system may employ logarithmic spacing, exponential progressions, or custom interval patterns that correspond to physiological response curves or user preference data. The maximum scale values may be extended or reduced based on application requirements, with consumer scales potentially reaching 2000 ml or higher for extended monitoring periods, while athletic scales may be customized to individual physiological limits determined through plateau detection algorithms. The system may also support dynamic scale adjustment, where interval spacing and maximum values are modified in real-time based on user response patterns, environmental conditions, or activity levels to optimize measurement precision and user compliance with hydration protocols.

The method 700 then proceeds to step 708, where it is determined whether the user has complied with the instruction to drink water. This determination may be made based on various indicators, such as the user's self-report, sensor data indicating water consumption, or changes in the user's PPG data indicating hydration. If the user has complied with the instruction, the method 700 continues to the next step. If the user has not complied with the instruction, the method 700 may repeat the instruction or take other actions to encourage the user to hydrate.

In some aspects, the method 700 may be implemented in a hydration monitoring system, such as the system 100 described herein. The system 100 may include a server and an interface-sensor system, which work together to collect hydration data, train a model for hydration prediction, and provide real-time hydration monitoring and alerting services to the user. The server may be configured to obtain PPG data samples, mark each sample with a label indicating a hydration level, and generate a model based on the labeled data. The interface-sensor system may include a PPG sensor and a user interface, which obtain PPG data, process the data through the model, and output an indicator based on the user's hydration state.

In step 710, incremental hydration PPG data is obtained for the hydration session. The PPG data may be obtained using a PPG sensor, such as the PPG sensor 125 described earlier. The PPG sensor may be configured to continuously monitor the user's PPG data and transmit the data to the user interface in real-time. The incremental hydration PPG data obtained at this stage may serve as a measure of the user's hydration status after consuming the instructed amount of water.

The method 700 then moves to a decision point at step 712, where it is determined if the routine should end. This determination may be made based on various factors, such as the user's hydration status, the time of day, or other relevant factors. If the routine does not end, the process returns to step 706 to provide another instruction to drink water. If the routine ends, the method 700 proceeds to step 714, where the hydration session data is provided to a server.

In some aspects, the determination of whether the routine should end may be based on the user's progression through the non-linear hydration scale and the detection of physiological plateau indicators. The system may evaluate whether the user has completed the population-based scale progression through the 0 ml, 500 ml, 700 ml, 800 ml, 900 ml, and 1000 ml increments, or whether individual-based training requires continuation beyond 1 liter. The decision may incorporate analysis of PPG data patterns to determine if additional fluid increments are needed to establish the user's maximum hydration state.

In some cases, the routine termination decision may consider whether the current hydration level represents the user's physiological maximum, as indicated by plateau detection in the PPG signal characteristics. For athletes or individuals in extreme conditions, the routine may continue beyond the standard 1-liter threshold with 200-250 ml increments until physiological indicators suggest that further fluid intake will not significantly alter the hydration assessment parameters. This approach may ensure that the training data captures the full range of the user's hydration response, from maximum dehydration to optimal hydration state.

In some aspects, the hydration sessions may be performed on successive days, as indicated in step 706. This allows the system to track the user's hydration status over multiple days and make more accurate predictions about the user's hydration needs. The hydration sessions may also be performed on days where the initial PPG data satisfies a baseline condition, ensuring that the user starts each hydration session from a similar hydration state.

In some cases, the system may be configured to mark samples with a first label based on an analysis of initial PPG data for a plurality of hydration sessions. This allows the system to generate a set of training data that reflects the user's hydration status over portions of hydration sessions and/or multiple hydration sessions. The first label may indicate a least hydrated state of the user over the plurality of hydration sessions, providing a baseline for the hydration monitoring process.

Following the provision of the hydration session data to the server, the method 700 proceeds to step 716, where it is determined if there is enough data to train or retrain the model at the server. This determination may be made based on various factors, such as the amount of data collected, the quality of the data, or other relevant factors. If there is not enough data (No branch), the method 700 returns to step 716 to determine if there is enough data. If there is enough data (Yes branch), the method 700 moves to step 718, where the model is trained or retrained based on the labeled data.

The training or retraining of the model may involve various machine learning techniques, such as supervised learning, unsupervised learning, reinforcement learning, or other suitable techniques. The model may be trained to recognize different hydration states based on PPG data, and to predict a user's hydration state based on a current PPG data sample.

Following the training or retraining, step 720 involves storing or updating the stored model for deployment. The model may be stored in a memory, such as memory 202 of the user interface 120 or memory 302 of the server 105. The stored model may be used for future hydration monitoring and prediction tasks.

In some aspects, the model may be updated periodically to improve its prediction accuracy. The updating process may involve retraining the model with new data, adjusting the model parameters, or other suitable methods. The updated model may provide more accurate and reliable hydration predictions, thereby improving the effectiveness of the hydration monitoring system.

FIG. 8 depicts a method 800 for hydration monitoring and model updating begins with step 802, where a current PPG data sample for a user is obtained via a PPG sensor. The PPG sensor, which may be part of an interface-sensor system such as the one described earlier, is configured to continuously monitor the user's PPG data and transmit the data to a user interface in real-time. The current PPG data sample may represent the user's PPG signal at a specific point in time, and may include various features of the PPG signal, such as its amplitude, frequency, phase, or other characteristics.

Following the acquisition of the current PPG data sample, the method 800 proceeds to step 804, where the current PPG data sample is processed through a model to obtain a hydration score for the user. The model, which may be the model 400 described earlier, is trained to interpret PPG data and determine hydration states. The model may use a variety of machine learning techniques to analyze the PPG data and generate the hydration score. The hydration score may be a numerical value that represents the user's hydration level, with higher scores indicating higher hydration levels.

Next, in step 806, a hydration state of the user is determined based on the hydration score. The hydration state may be one of several predefined states, such as a dehydrated state, a partially dehydrated state, a partially hydrated state, or a fully hydrated state. The user interface device may determine the user's hydration state by comparing the hydration score to a set of thresholds associated with the predefined states. For example, if the hydration score falls within a range associated with the partially hydrated state, the user interface device may determine that the user is in the partially hydrated state.

Following the determination of the hydration state, step 808 involves outputting an indicator based on the hydration state. The indicator may be a visual, auditory, or tactile signal that informs the user of their hydration state. For example, the indicator may be a color-coded bar on the user interface, with different colors representing different hydration states. The indicator may also include a recommendation for the user to consume a certain amount of fluid to reach a desired hydration state.

In some aspects, the user interface device may be further configured to determine that the hydration state indicates dehydration and generate an alert to the user to increase fluid intake. This alert may be a visual, auditory, or tactile signal that prompts the user to drink water or consume other fluids. The alert may be particularly useful in situations where the user is engaged in physical activity or exposed to hot weather, which can increase the risk of dehydration.

In some cases, the user interface device may be further configured to track the user's fluid intake over time, correlate the fluid intake with changes in the hydration state, and provide personalized hydration recommendations based on the correlation. This feature may allow the user to better manage their hydration levels and prevent dehydration or overhydration. The personalized hydration recommendations may take into account various factors, such as the user's body weight, activity level, and environmental conditions.

In some aspects, the user interface device may be further configured to detect a sudden change in the hydration state and trigger an emergency alert if the sudden change exceeds a predetermined threshold. This feature may be particularly useful in situations where the user is at risk of severe dehydration or overhydration, which can lead to serious health complications. The emergency alert may prompt the user to seek immediate medical attention or take other urgent actions to address the hydration imbalance.

The method 800 may continue with step 810, where user feedback on the hydration state is received. The user feedback may be obtained through the user interface 120 and may include the user's subjective assessment of their hydration state, their perceived accuracy of the hydration state determined by the system, or other relevant information. The user feedback may be used to adjust the model parameters, improve the accuracy of the hydration state determination, or tailor the hydration recommendations to the user's individual needs.

Following the receipt of user feedback, the method 800 proceeds to step 812, where the data and user feedback are packaged and provided to a server. The server, which may be the server 105 described earlier, is configured to receive the packaged data and user feedback, process the data, and manage communications with the user interface 120. The server 105 may use the received data and user feedback to update the model, generate new hydration recommendations, or perform other operations.

In step 814, at the server, the server may store the data and user feedback. In some cases, a decision point is reached to determine if there is enough data to train or retrain the model. This determination may be made based on various factors, such as the amount of data collected, the quality of the data, the diversity of the data, or other relevant factors. If there is not enough data, the method may determine that there is not enough data. If there is enough data (e.g., a training trigger), the method moves to step 816, where the model is trained or retrained based on the data from the population.

Following the training or retraining, step 818 involves storing or updating the stored model for deployment. The model may be stored in a memory, such as memory 202 of the user interface 120 or memory 302 of the server 105. The stored model may be used for future hydration monitoring and prediction tasks.

Finally, in step 820, the trained or retrained model(s) are provided, back to the user's device for future hydration monitoring. This step completes the feedback loop, allowing for continuous improvement of the hydration monitoring process.

In some aspects, the method 800 represents a cyclical process of data collection, analysis, user feedback, and model improvement. It incorporates both individual user data and population data to enhance the accuracy of hydration state determination over time.

In case where user feedback on the hydration state is received, the user feedback may be obtained through the user interface 120 and include the user's subjective assessment of their hydration state, their perceived accuracy of the hydration state determined by the system, or other relevant information. The user feedback may be used to adjust the model parameters, improve the accuracy of the hydration state determination, or tailor the hydration recommendations to the user's individual needs.

When the data and user feedback are packaged and provided to a server, the server, which may be the server 105 described earlier, may be configured to receive the packaged data and user feedback, process the data, and manage communications with the user interface 120. The server 105 may use the received data and user feedback to update the model, generate new hydration recommendations, or perform other operations.

In some aspects, the method 800 represents a cyclical process of data collection, analysis, user feedback, and model improvement. It incorporates both individual user data and population data to enhance the accuracy of hydration state determination over time. The method 800 may be implemented in a hydration monitoring system, such as the system 100 described earlier. The system 100 may include a server and an interface-sensor system, which work together to collect hydration data, train a model for hydration prediction, and provide real-time hydration monitoring and alerting services to the user. The server may be configured to obtain PPG data samples, mark each sample with a label indicating a hydration level, and generate a model based on the labeled data. The interface-sensor system may include a PPG sensor and a user interface, which obtain PPG data, process the data through the model, and output an indicator based on the user's hydration state.

In some aspects, the model used in the hydration monitoring system may be a machine learning model. Machine learning models are computational models that are capable of learning patterns from data and making predictions or decisions without being explicitly programmed to perform the task. In the context of the hydration monitoring system, the machine learning model may be trained to recognize different hydration states based on PPG data and to predict a user's hydration state based on a current PPG data sample.

In some cases, the machine learning model may be a user-specific model. A user-specific model is a model that is tailored to a specific user based on the user's individual characteristics and data. This may include the user's body weight, activity level, hydration habits, and other relevant factors. The user-specific model may be more accurate and personalized than a general model that is trained on population data.

In some aspects, the user-specific model may be a calibrated version of a population model derived from the training data. The population model may be a general model that is trained on a large set of PPG data samples from a population of users. The population model may be calibrated to a specific user by adjusting the model parameters based on the user's individual data. This calibration process may improve the accuracy and personalization of the model.

In some cases, the machine learning model may use data associated with the user as a self-reference for determination of the hydration state. This self-referential data may include the user's past hydration states, fluid intake, activity levels, and other relevant data. The machine learning model may use this self-referential data to learn the user's individual hydration patterns and make more accurate hydration predictions.

In some aspects, the machine learning model may be configured to receive input PPG data and determine an output hydration score based on the input PPG data. The input PPG data may be a current PPG data sample obtained from the user. The output hydration score may be a numerical value that represents the user's hydration level, with higher scores indicating higher hydration levels. The machine learning model may use various machine learning techniques to analyze the input PPG data and generate the output hydration score.

In some aspects, the machine learning model may operate on a plurality of parameters based on at least a first sample and a second sample. These parameters may include various features or characteristics of the PPG data, such as the amplitude, frequency, phase, or other attributes of the PPG signal. The parameters may also include temporal features, such as the time interval between consecutive PPG samples, the duration of a PPG cycle, or other time-related features. The parameters may further include spectral features, such as the power spectrum, the frequency spectrum, or other spectral characteristics of the PPG signal.

In some cases, the plurality of parameters may include statistics of PPG traces derived from the PPG data. These statistics may include various statistical measures or descriptors of the PPG traces, such as the mean, median, mode, variance, standard deviation, skewness, kurtosis, or other statistical measures. The statistics may provide a quantitative description of the distribution, variability, or other characteristics of the PPG traces, which may be useful for determining the user's hydration status.

In some aspects, the plurality of parameters may include user demographic data. The user demographic data may include various demographic characteristics of the user, such as the user's age, gender, body weight, body mass index (BMI), ethnicity, or other demographic factors. The user demographic data may be used to personalize the hydration monitoring process and improve the accuracy of the hydration state determination.

In some cases, the plurality of parameters may include user activity data and user sleep data. The user activity data may include various measures or indicators of the user's physical activity, such as the number of steps taken, the distance traveled, the calories burned, the intensity of the activity, or other activity measures. The user sleep data may include various measures or indicators of the user's sleep patterns, such as the duration of sleep, the quality of sleep, the sleep stages, or other sleep measures. The user activity data and user sleep data may be used to account for the effects of physical activity and sleep on the user's hydration status.

In some embodiments, the model may comprise a data preprocessing block configured to perform denoising and short-time Fourier transform (STFT) on the current PPG data sample. The denoising operation may involve various noise reduction techniques, such as filtering, smoothing, or other denoising methods, to remove or reduce the noise in the PPG data. The STFT operation may involve applying a Fourier transform to short segments of the PPG data to obtain a time-frequency representation of the PPG signal. The STFT operation may allow the model to capture both temporal and spectral features of the PPG signal, which may be crucial for accurately determining the user's hydration status.

In some cases, the method 800 may use the model 400. The model 400 may include a series of convolutional layers that are designed to extract spatial features from the input data. These convolutional layers apply a set of filters to the input data, which can capture local patterns in the data. The filters are learned during the training process, allowing the model to adapt to the specific characteristics of the PPG data.

In some aspects, the first convolutional layer 403 of the model 400 may be configured to apply first filters of a first predetermined size with ReLU (Rectified Linear Unit) activation. The ReLU activation function introduces non-linearity into the model, allowing it to capture complex patterns in the data. The first convolutional layer 403 may also be configured to perform batch normalization and 3D max pooling on its output. Batch normalization is a technique that normalizes the input data for each mini-batch, which can accelerate and stabilize the training process. 3D max pooling is an operation that reduces the spatial dimensions of the data by taking the maximum value over a sliding window, which can help to reduce overfitting and improve computational efficiency.

In some cases, the model 400 may include a second convolutional layer 404 configured to apply second filters of a second predetermined size with ReLU activation. Like the first convolutional layer 403, the second convolutional layer 404 may also be configured to perform 3D max pooling on its output. The second convolutional layer 404 may extract more complex features from the data, building upon the features extracted by the first convolutional layer 403.

In some aspects, the model 400 may further comprise a third convolutional layer 405 configured to apply third filters of a third predetermined size with padding and ReLU activation. Padding is a technique that adds extra pixels around the input data, allowing the convolutional operation to be applied to the border pixels of the data. This can help to preserve the spatial dimensions of the input feature maps, ensuring that valuable information at the borders of the data is not lost.

In some cases, the model 400 may be configured to increase the filter count in each successive convolutional layer. This can facilitate the extraction of more intricate features from the PPG data, allowing the model to capture a wider range of patterns in the data. The specific configuration of the convolutional layers, including the size and number of filters and the use of padding and ReLU activation, may be determined based on the specific requirements of the hydration monitoring task.

Following the convolutional layers, the data in the model 400 is processed by a dense layer 406. The dense layer 406, in some aspects, may include a plurality of neurons and utilize ReLU activation. The dense layer 406 applies fully connected neural network operations to further process the extracted features. These layers integrate powerful non-linear combinations of features derived from preceding convolutional layers, aiding the model in learning complex dependencies.

Subsequently, the output from the dense layers is used to generate a class prediction 408. This prediction represents the model's assessment of the hydration status based on the input PPG data. The class prediction 408 is generated using a sigmoid activation function, which compresses the output to a range of [0, 1], making it ideal for binary classification.

In some cases, the model 400 may be configured to process the current PPG data sample through a series of convolutional layers, each applying an increasing number of filters. This progressive increase in filter count across the convolutional layers, from the first convolutional layer 403 to the third convolutional layer 405, facilitates the extraction of more intricate features from the PPG data. This feature extraction process allows the model to capture a wider range of patterns in the data, thereby enhancing its ability to accurately determine the user's hydration state.

In some aspects, the model 400 may be configured to operate on a plurality of parameters based on at least a first sample and a second sample. These parameters may include various features or characteristics of the PPG data, such as the amplitude, frequency, phase, or other attributes of the PPG signal. The parameters may also include temporal features, such as the time interval between consecutive PPG samples, the duration of a PPG cycle, or other time-related features. The parameters may further include spectral features, such as the power spectrum, the frequency spectrum, or other spectral characteristics of the PPG signal.

In some cases, the model 400 may be configured to use padding in the third convolutional layer 405 to preserve the spatial dimensions of the input feature maps. This padding technique can help to ensure that valuable information at the borders of the data is not lost during the convolution operation. This can be particularly important when processing PPG data, as the borders of the data may contain crucial information about the user's hydration status.

In some aspects, the model 400 may be configured to output a hydration score between 0 and 1, with values closer to 0 indicating dehydration and values closer to 1 indicating hydration. This hydration score can provide a quantitative measure of the user's hydration level, allowing the system to accurately determine the user's hydration state and provide appropriate hydration recommendations.

In some embodiments, the model 400 may be specifically configured and architected to process the PPG data in a manner that enhances the accuracy of hydration state determination. The model 400 may include a series of convolutional layers, each applying an increasing number of filters. For instance, the first convolutional layer 403 may apply 12 filters, the second convolutional layer 404 may apply 24 filters, and the third convolutional layer 405 may apply 48 filters. This progressive increase in filter count across the convolutional layers facilitates the extraction of more intricate features from the PPG data, thereby enhancing the model's ability to accurately determine the user's hydration state.

In some cases, the model 400 may utilize ReLU activation in all layers except for the output layer. The ReLU activation function introduces non-linearity into the model, allowing it to capture complex patterns in the data. This non-linearity can be crucial for accurately determining hydration states, as the relationship between PPG data and hydration levels may not be linear.

In some aspects, the model 400 may use sigmoid activation in the output layer. The sigmoid activation function compresses the output to a range of [0, 1], making it ideal for binary classification tasks such as determining whether a user is dehydrated or hydrated. This compressed output range can provide a clear and interpretable hydration score, facilitating user understanding and engagement.

In some embodiments, the model 400 may perform 3D max pooling after each convolutional layer. The 3D max pooling operation reduces data dimensionality by capturing the most significant features, thus mitigating the risk of overfitting and improving computational efficiency. This operation can be particularly beneficial when processing high-dimensional PPG data, as it allows the model to focus on the most relevant features for hydration state determination.

In some cases, the model 400 may flatten the output of the convolutional layers before passing it to a dense layer. This flattening operation transforms the multi-dimensional output of the convolutional layers into a one-dimensional vector, which can be more easily processed by the subsequent dense layer. This operation can help to streamline the data processing pipeline, enhancing the efficiency and scalability of the model.

In some embodiments, the model 400 may include additional technical features that enhance the hydration monitoring process. For instance, the model 400 may be configured to use batch normalization after the first convolutional layer 403. Batch normalization is a technique that normalizes the input data for each mini-batch, which can accelerate and stabilize the training process. This can be particularly beneficial when processing large amounts of PPG data, as it can help to prevent overfitting and improve the generalization performance of the model.

In some cases, the model 400 may be configured to use padding in the third convolutional layer 405. Padding is a technique that adds extra pixels around the input data, allowing the convolutional operation to be applied to the border pixels of the data. This can help to preserve the spatial dimensions of the input feature maps, ensuring that valuable information at the borders of the data is not lost during the convolution operation. This can be particularly important when processing PPG data, as the borders of the data may contain crucial information about the user's hydration status.

In some aspects, the model 400 may be configured to output a hydration score between 0 and 1, with values closer to 0 indicating dehydration and values closer to 1 indicating hydration. This hydration score can provide a quantitative measure of the user's hydration level, allowing the system to accurately determine the user's hydration state and provide appropriate hydration recommendations. The hydration score may be generated using a sigmoid activation function in the output layer, which compresses the output to a range of [0, 1], making it ideal for binary classification tasks such as determining whether a user is dehydrated or hydrated.

In some embodiments, the model 400 may be configured to process the current PPG data sample through a neural network architecture comprising at least three convolutional layers, one dense layer, and one output layer. This architecture allows the model to extract complex features from the PPG data and make accurate predictions about the user's hydration state. The convolutional layers may be configured to apply a series of filters to the input data, capturing local patterns in the data. The dense layer may integrate powerful non-linear combinations of features derived from the convolutional layers, aiding the model in learning complex dependencies. The output layer may generate the final hydration score based on the processed data.

In some cases, the PPG sensor 125 may be configured to obtain the current PPG data sample using green light with a wavelength between 525 nm and 540 nm. This specific wavelength range can provide optimal sensitivity for detecting changes in blood volume, which can be indicative of changes in hydration status. The PPG sensor 125 may be a wearable device, such as a wristband or a watch, that the user can wear throughout the day. The PPG sensor 125 may be configured to continuously monitor the user's PPG data and transmit the data to the user interface 120 in real-time, enabling real-time hydration monitoring and alerting.

In some aspects, the system may utilize a labeling system for hydration levels. The set of labels may include at least five labels indicating different hydration levels. These labels may correspond to 20% intervals on a hydration scale from 0 to 100%. For instance, the first label may indicate a hydration level of 0-20%, the second label may indicate a hydration level of 20-40%, and so on, up to the fifth label indicating a hydration level of 80-100%. This labeling system allows for a granular representation of the user's hydration status, providing more detailed and accurate hydration monitoring.

In some cases, the first label may be used to indicate a least hydrated state of the user over a plurality of hydration sessions. This least hydrated state may correspond to a state of dehydration, where the user's body has lost a significant amount of water and needs to replenish its fluid levels. The first label may be assigned to PPG data samples obtained during this state, providing a baseline for the hydration monitoring process.

In some aspects, the system may utilize a wearable device for monitoring the user's hydration status. The wearable device may include a user interface and a PPG sensor. The PPG sensor may be configured to continuously monitor the user's PPG data and transmit the data to the user interface in real-time. The user interface may be configured to obtain the current PPG data sample from the PPG sensor, process the data through the model, and output an indicator based on the user's hydration state.

In some cases, the wearable device may be a smartwatch. The smartwatch may be worn on the user's wrist, allowing for convenient and unobtrusive monitoring of the user's PPG data. The smartwatch may include a display for presenting the hydration status indicator to the user, as well as controls for interacting with the hydration monitoring system. The smartwatch may also include other features, such as a heart rate monitor, a step counter, or a sleep tracker, providing a comprehensive health monitoring solution.

In some aspects, the server 105 may be further configured to aggregate anonymized hydration data from multiple users and generate population-level hydration insights based on the aggregated data. This feature allows the system to analyze hydration trends and patterns across a large population of users, providing valuable insights into hydration behaviors and needs at a population level.

The aggregated hydration data may include various types of data, such as PPG data, hydration scores, hydration states, user feedback, and other relevant data. The data may be anonymized to protect the privacy of the users, ensuring that individual user identities are not disclosed in the aggregated data.

The server 105 may use various data aggregation techniques to combine the hydration data from multiple users. For instance, the server 105 may calculate averages, medians, modes, or other statistical measures of the hydration data. The server 105 may also group the data by certain characteristics, such as age, gender, activity level, or other demographic or behavioral factors.

The population-level hydration insights generated by the server 105 may include various types of insights, such as trends in hydration levels over time, correlations between hydration levels and other factors, distributions of hydration states, or other insights. These insights can provide valuable information for researchers, healthcare providers, fitness professionals, and others interested in understanding and improving hydration behaviors and outcomes at a population level.

In some cases, the server 105 may also provide the population-level hydration insights to the user interface 120. The user interface 120 may display these insights to the user, providing them with a broader context for their personal hydration status. For instance, the user interface 120 may show the user how their hydration level compares to the average hydration level of the population, or how their hydration behaviors align with population trends. This feature can help users understand their hydration status in a broader context, potentially motivating them to improve their hydration habits.

In some aspects, the server 105 may periodically update the population-level hydration insights based on new hydration data from the users. This allows the system to provide up-to-date insights that reflect the most recent hydration behaviors and trends in the population. The server 105 may also use the updated insights to refine the model, improving the accuracy of hydration state determination and prediction over time.

### 7. Maximum Dehydration and Hydration

The present disclosure provides methods for hydration monitoring that work together to provide accurate and personalized hydration assessment. These methods include a method for determining a user's maximum dehydration state and a method for determining a user's maximum hydration state. These methods are integral components of the overall hydration monitoring system, which comprises a server and an interface-sensor system.

The server in the hydration monitoring system is configured to obtain a set of photoplethysmogram (PPG) data samples for a set of population users. This data collection process forms the foundation for developing a robust hydration assessment model. The server is further configured to mark each PPG data sample with a selected label from a set of labels to obtain a set of training data. The selected label indicates a hydration level of a population user at a specific time corresponding to when a respective PPG data sample was obtained. This labeling process is crucial for creating a comprehensive dataset that represents various hydration states.

The set of labels used in the system includes at least two labels indicating different hydration levels. In some cases, these labels may include a first label for dehydration and a second label for hydration. This binary classification allows the system to distinguish between hydrated and dehydrated states, providing a clear basis for hydration assessment.

In some aspects, the system may utilize a more granular labeling approach that includes intermediate states between maximum hydration and maximum dehydration. The set of labels may include multiple categories representing various hydration levels along a continuous spectrum. For example, the labels may include “severely dehydrated.” “mildly dehydrated.” “adequately hydrated.” “well hydrated.” and “optimally hydrated.”

The model may be trained on this expanded set of labeled data, allowing it to recognize and classify a wider range of hydration states. By incorporating these intermediate labels, the model may develop a more nuanced understanding of the relationship between PPG data patterns and different degrees of hydration.

In some cases, the model may be designed to output a continuous hydration score rather than discrete categories. This score may represent a point along the spectrum between maximum dehydration and maximum hydration. The model may learn to infer these gradations by recognizing subtle differences in the PPG data that correspond to small changes in hydration levels.

The system may use techniques such as regression analysis or ordinal classification to enable the model to predict these finer gradations. In some implementations, the model may employ a softmax activation function in the output layer, which can provide probabilities for each hydration state. These probabilities may be used to infer the user's position on the hydration spectrum.

In some aspects, the system may combine the discrete labels with continuous scoring. For instance, the model may output both a categorical prediction (e.g., “mildly dehydrated”) and a numerical score (e.g., 3.7 on a scale of 1 to 10). This approach may provide users with both an easily interpretable category and a more precise indication of their hydration status.

The ability to infer gradations between maximum hydration and maximum dehydration may allow for more personalized and actionable hydration recommendations. For example, the system may suggest different fluid intake amounts or hydration strategies based on the user's specific position on the hydration spectrum, rather than using a one-size-fits-all approach for broad categories.

Using this labeled dataset, the server is configured to generate a model based on the set of training data. This model serves as the core component for analyzing PPG data and determining hydration levels in real-time. The model may be trained to recognize patterns in PPG data that correspond to different hydration states, enabling accurate hydration assessment for individual users.

The method for determining a user's maximum dehydration state involves monitoring the user's PPG data over time, particularly during periods when the user is likely to be in a dehydrated state, such as upon waking after a night's sleep. This method helps establish an estimate for the user's most dehydrated state, which can be used as a reference point for subsequent hydration assessments.

Complementing this, the method for determining a user's maximum hydration state involves monitoring the user's PPG data as they consume fluids and reach a state of plateaued hydration. This method helps identify the PPG characteristics associated with the user's fully hydrated state, providing an upper bound for hydration assessment.

By combining these methods, the hydration monitoring system can provide a personalized and comprehensive assessment of a user's hydration status. The system may use the maximum dehydration state and maximum hydration state as reference points to accurately interpret PPG data and determine the user's current hydration level. This approach allows for more precise hydration monitoring and can help users maintain optimal hydration levels throughout their daily activities.

In some cases, the interface-sensor system may continuously collect PPG data from the user and transmit this data to the server for analysis. The interface-sensor system or the server may then apply the trained model to this real-time data to provide ongoing hydration assessments. This continuous monitoring capability enables the system to detect changes in hydration status promptly and provide timely recommendations for fluid intake.

The combination of these methods and components creates a comprehensive hydration monitoring system that can adapt to individual users' hydration patterns and provide personalized hydration guidance. This system may offer significant benefits in various applications, from improving athletic performance to managing health conditions where hydration plays a crucial role.

The present disclosure provides a method for collecting a maximum dehydration state and training a model to monitor and analyze hydration levels. A method 900 may be implemented to determine a user's maximum dehydration point, alert the user to deviations from their hydration state, and update the maximum dehydration value as needed.

In some cases, a step 902 of the method 900 may involve determining that a user has woken up from a threshold sleep amount. This determination may be made using various techniques. For example, the method 900 may utilize data from other sensors or APIs, such as sleep tracking functionality, to determine the wake-up time for establishing a dehydration baseline.

In some cases, the method 900 may use a transition from resting heart rate to normal daily heart rate to correlate with photoplethysmogram (PPG) shape for determining the dehydration state. A step 904 may involve obtaining initial PPG data before the user hydrates. This initial PPG data may represent the point of maximum dehydration when a user wakes up in the morning.

A step 906 of the method 900 may involve monitoring hydration levels using PPG data and a model. The model may be trained to recognize patterns in PPG data that correspond to different hydration states. In some cases, the model may be updated to use the maximum dehydration point to improve inference of the user's hydration state.

A step 908 may involve determining if there are deviations from a previous state. If deviations are detected, a step 910 may involve outputting alerts for the detected deviation. These alerts may serve as preventative warnings as the hydration level decreases during activities. For example, if the user's hydration level drops significantly during exercise, the method 900 may generate an alert prompting the user to increase fluid intake.

In some cases, a step 912 may involve determining if the deviation represents a new maximum dehydration. The method 900 may adjust the baseline dehydration point based on various factors. For example, alcohol consumption, intense exercise, or exposure to extreme conditions may lead to a lower hydration state than the typical morning dehydration. If the deviation is determined to be a new maximum dehydration, a step 914 may involve updating the maximum dehydration value.

By continuously monitoring and updating the maximum dehydration value, the method 900 may provide more accurate and personalized hydration monitoring. The updated maximum dehydration value may be used to recalibrate the model, allowing for more precise detection of deviations from the user's normal hydration state.

In some cases, the method 900 may use the updated maximum dehydration value to adjust the thresholds for generating alerts. For example, if the user's maximum dehydration point has shifted due to changes in their lifestyle or environment, the method 900 may adjust the alert thresholds accordingly to ensure that warnings are provided at appropriate times.

The method 900 may operate in a continuous loop, returning to step 906 to continue monitoring hydration levels after updating the maximum dehydration value or if no deviations are detected. This continuous monitoring allows for real-time tracking of the user's hydration state and prompt detection of any significant changes.

The present disclosure provides a method for determining a user's maximum hydration state over a period of 1-N days using incremental increases in water consumption. This method may be implemented as part of a hydration monitoring system that includes an interface-sensor system with a photoplethysmogram (PPG) sensor and a user interface.

A method 1000 for determining a user's hydration levels may begin with a step 1002 where the system determines that a user has woken up from a threshold sleep amount on day 1. This determination may be made using various sensors or user input through the user interface.

In a step 1004, the system may obtain initial PPG data before the user hydrates. A PPG sensor may be configured to obtain a current PPG data sample for the user. This initial PPG data may serve as a baseline for the user's hydration level upon waking.

The method 1000 may proceed to a step 1006, where instructions are provided to drink a first amount of water. These instructions may be displayed on the user interface, which may be configured to indicate a hydration state of the user based on the current PPG data sample.

In a step 1008, the system may determine user compliance with the drinking instruction. This determination may be made through user input or through sensors that detect fluid consumption.

A step 1010 may involve obtaining incremental hydration PPG data. The PPG sensor may continuously collect PPG data samples as the user's hydration level changes due to fluid consumption.

At a decision step 1012, the system may check for plateau detection. Plateau detection may involve analyzing the PPG data to determine if the PPG shape has stopped changing, which may indicate that the user has reached their maximum hydration point.

In some aspects, plateau detection may involve analyzing the PPG data using various statistical measures and techniques to determine if the PPG shape has plateaued, indicating that the user has reached their maximum hydration point. The system may employ multiple approaches to detect this plateau, considering both short-term and long-term trends in the PPG data.

For plateau detection within the most recently obtained signal, the system may calculate and monitor several statistical measures, including:
1. Moving average: The system may compute a moving average of the PPG signal amplitude over a specified time window. A stabilization in this moving average may indicate a plateau.
2. Standard deviation: The system may calculate the standard deviation of the PPG signal amplitude. A decrease in standard deviation below a certain threshold may suggest that the signal has stabilized.
3. Coefficient of variation: The system may compute the coefficient of variation (CV) of the PPG signal amplitude. A reduction in CV may indicate decreased variability and potential plateau.
4. Slope analysis: The system may perform a linear regression on recent PPG data points and analyze the slope. A slope approaching zero may suggest a plateau.
5. Frequency domain analysis: The system may apply Fourier transform to the PPG signal and monitor changes in the frequency spectrum. Stabilization in the dominant frequencies may indicate a plateau.

For example, the system may determine that a plateau has been reached if the moving average of the PPG signal amplitude remains within +2% for a period of 15 minutes, the standard deviation decreases by 50% compared to the initial measurement, and/or the slope of the linear regression line falls below 0.01.

In some cases, the system may also compare the most recently obtained signal to data from previous hydration sessions to detect a plateau. This comparison may involve:
1. Day-to-day correlation: The system may calculate the correlation coefficient between the current PPG signal and the signal from the same time point in the previous day's hydration session. A high correlation may indicate a similar hydration state.
2. Relative change analysis: The system may compute the percentage change in PPG signal characteristics (e.g., amplitude, frequency) from the previous day's maximum hydration point. A small relative change may suggest that the current hydration state is approaching the previous day's maximum.
3. Pattern matching: The system may use dynamic time warping or other pattern matching algorithms to compare the current PPG signal shape to the shape observed at the plateau in previous sessions.
4. Statistical distance measures: The system may calculate statistical distance measures such as Kullback-Leibler divergence or Jensen-Shannon divergence between the current PPG signal distribution and the distribution observed at the plateau in previous sessions.

For instance, the system may determine that a plateau has been reached if the correlation coefficient between the current PPG signal and the previous day's signal at maximum hydration exceeds 0.95, the relative change in signal amplitude is less than 3%, and/or the Jensen-Shannon divergence between the current and previous signal distributions is below 0.05.

In some aspects, the system may combine multiple plateau detection methods, assigning weights to each method based on their historical accuracy or relevance to the specific user. This multi-faceted approach may enhance the robustness and reliability of plateau detection, accounting for individual variations in hydration response and day-to-day fluctuations in physiological state.

In some aspects, the system may implement an incremental hydration approach to determine the user's maximum hydration state. This process may involve collecting data, instructing the user to consume specific amounts of water, obtaining sensor data, and analyzing the PPG signal to determine if a hydration plateau has been reached over successive days.

The system may begin by instructing the user to drink a fixed amount of fluid every morning, such as 500 ml. The PPG sensor may continuously register the shape of the PPG signal throughout this process.

On the first day, after the user consumes 250 ml of fluid, the system may detect changes in the PPG shape indicating improved hydration. The algorithm may calculate an appropriate hydration percentage and classify the user's hydration state based on these changes.

On the second day, the system may instruct the user to consume 500 ml of fluid. The hydration analysis may show improvement compared to the first day, reflected in changes to the PPG signal shape. The system may update its analysis of the PPG data and adjust the user's hydration class and percentage accordingly.

The process may continue on the third day, with the system instructing the user to consume 750 ml of fluid. The hydration outcome may show further improvement, with corresponding changes in the PPG signal shape. The system's analysis may reflect this enhanced hydration status.

On the fourth day, the system may instruct the user to consume 1000 ml of fluid. At this point, or potentially on the following day after consuming 1250 ml, the system may detect that the PPG signal shape has stopped changing significantly. The analysis may reflect a consistent hydration status compared to the previous day, indicating that a plateau has been reached.

In some cases, the amount of fluid required to reach this plateau may vary depending on the individual user. For users who start from a severely dehydrated baseline, the system may need to continue the incremental process for additional days or with larger fluid amounts.

Once the plateau is detected, the system may determine that the user has reached their ideal hydration state. At this point, the system may recognize that additional fluid consumption will not significantly improve the user's hydration status, as excess fluids will be excreted by the kidneys.

This plateau detection process may allow the system to establish a personalized maximum hydration state for each user, which can serve as a reference point for future hydration monitoring and recommendations.

If plateau is detected, the method 1000 may proceed to a step 1014, where hydration session data is provided to a server and/or the user (e.g., via the interface). This data may include the PPG samples collected throughout the hydration process, as well as information about the user's fluid consumption.

If plateau is not detected, the method 1000 may proceed to a step 1016 to determine the day of 2-N day of incremental increase. This step may involve planning for subsequent days of the hydration level determination process.

In a step 1018, the system may determine a second amount and time frame for the user to consume water. This determination may be based on the user's response to the previous day's fluid consumption and the PPG data collected.

The method 1000 may then move to a step 1020, where instructions are provided to drink the second amount of water and confirm user compliance. These instructions may be displayed on the user interface.

In a step 1022, the system may obtain incremental hydration PPG data, after which the process may return to the decision step 1012 to check for plateau detection.

The user interface may be configured to obtain a model for hydration analysis. This model may be used to process the current PPG data sample and obtain a hydration score for the user. Based on this hydration score, the user interface may determine a hydration state of the user.

Throughout the process, the user interface may be configured to output an indicator based on the hydration state. This indicator may be displayed on the user interface, providing real-time feedback to the user about their hydration status.

In some cases, the system may update the model based on the user's maximum hydration data collected during this process. By incorporating this personalized data, the model may improve its inference capabilities for the specific user, leading to more accurate hydration state determinations in future assessments.

This method for determining a user's maximum hydration state may provide valuable personalized data that can enhance the accuracy of the hydration monitoring system. By establishing a user-specific ideal hydration point, the system may offer more precise hydration recommendations and alerts tailored to the individual user's physiology.

### 8. Model Enhancements

The hydration monitoring system described herein incorporates advanced technical enhancements that significantly improve the accuracy, efficiency, and personalization of hydration assessment. These enhancements build upon the foundational architecture comprising the server 105 and interface-sensor system 115, extending the system's capabilities through sophisticated signal processing techniques, multi-modal sensor integration, and adaptive training methodologies.

The enhanced system addresses key limitations in traditional hydration monitoring approaches by implementing targeted improvements across multiple technical domains. Signal processing enhancements optimize the extraction and analysis of physiologically relevant information from PPG data, while expanded sensor integration provides complementary physiological measurements that enhance hydration state determination accuracy. Advanced training methodologies enable the system to adapt to diverse user populations and individual physiological characteristics, providing both immediate usability and personalized precision.

These technical advances represent a comprehensive evolution of the hydration monitoring system, transforming it from a single-parameter PPG analysis tool into a sophisticated multi-modal physiological assessment platform. The enhancements maintain compatibility with the existing system architecture while extending functionality to support advanced applications including athletic performance monitoring, medical condition management, and extreme environmental condition assessment. These enhancements collectively enable more accurate, efficient, and personalized hydration assessment while maintaining the system's core advantages of non-invasive monitoring and real-time feedback.

The hydration monitoring system may implement a sophisticated two-tier training methodology that balances broad applicability with personalized accuracy through coordinated operation of the server 105 and interface-sensor system 115. This approach recognizes that different user populations have varying requirements for hydration monitoring precision and training investment, enabling flexible deployment strategies that optimize both immediate usability and long-term accuracy.

The population-based approach leverages the server 105 architecture to develop a general model trained on PPG data from multiple subjects across diverse demographic and physiological characteristics. The platform data manager 302B aggregates PPG data samples from the set of population users, while the platform model manager 302A coordinates the training process across multiple interface-sensor systems 115. This distributed data collection approach enables the model generator 302D to process comprehensive datasets representing varied physiological responses to hydration changes.

The server 105 implements the standardized routine described in the training methodology through coordination with multiple interface-sensor systems 115. Each system's PPG sensor 125 and other sensors 130 contribute synchronized multi-parameter data during incremental hydration sessions, generating comprehensive training datasets that capture population-wide hydration response patterns. The routine manager 302C orchestrates these data collection sessions across the user population, ensuring consistent protocol implementation while accommodating individual scheduling and compliance variations.

The population-based model offers immediate deployment capability through the device manager 302E, which distributes the trained model to new interface-sensor systems 115 without requiring extensive individual calibration periods. The model manager 202A in each user interface 120 can immediately begin processing current PPG data samples through the population model, providing baseline hydration monitoring functionality from the first use session.

Population model training incorporates the updated non-linear hydration scale and BMI correlation system through the platform data manager 302B, which processes training data that reflects the 0 ml, 500 ml, 700 ml, 800 ml, 900 ml, and 1000 ml increment progression. The server 105 ensures representative coverage of different hydration states and body compositions by collecting data from users across BMI ranges, with fluid intake protocols adjusted according to the adjusted amount for individuals with different BMIs.

The system may implement mathematical correlations that utilize various anthropometric and physiological inputs to calculate personalized fluid requirements for different operational phases. In some cases, the system may employ Body Mass Index (BMI) calculations as primary inputs for determining baseline fluid adjustments, where BMI values are processed through algorithms that correlate body composition with blood volume requirements during training data collection routines. The system may utilize blood volume (BV) estimations calculated through Nadler equations that incorporate height and weight parameters, where blood volume in liters serves as an input for determining individualized fluid intake recommendations during hydration protocols. In some aspects, the system may process plasma volume (PV) calculations derived from blood volume and hematocrit measurements to refine fluid requirement estimations, particularly for applications targeting athletes or individuals with elevated muscle mass who demonstrate proportionally larger blood volume increases compared to individuals with higher adipose tissue ratios. The system may apply these calculated values differently across operational contexts, using BMI-adjusted fluid volumes during training data collection phases to ensure representative physiological sampling, while employing blood volume-based adjustments during real-time hydration inference to provide personalized recommendations that account for individual cardiovascular capacity and fluid distribution characteristics. In some cases, the system may incorporate lean body mass estimations as supplementary inputs that enhance fluid requirement calculations for athletic applications, recognizing that blood volume correlates more closely with lean tissue mass than total body weight, thereby enabling more precise hydration guidance for users with varying body composition profiles.

The system may implement precise mathematical correlations for fluid intake adjustments based on individual physiological characteristics, as demonstrated through comparative analysis of users with different body compositions. For example, considering two males of identical height (170 cm) with 40% hematocrit but different weights of 63 kg versus 90 kg, the system may calculate corresponding BMI values of 21.8 versus 31.1, blood volumes of 4.4 liters versus 5.3 liters, and plasma volumes of 2.8 liters versus 3.5 liters using established physiological formulas. The system may apply these calculated differences to determine that the individual with higher BMI requires proportionally adjusted fluid intake during training protocols, reflecting the 20% increase in blood volume (from 4.4 L to 5.3 L) and 25% increase in plasma volume (from 2.8 L to 3.5 L) that corresponds to their physiological characteristics. In some cases, the system may recognize that larger blood volume increases occur predominantly in individuals with elevated muscle mass rather than adipose tissue, enabling differentiated scaling approaches where consumer-oriented protocols may utilize BMI-based adjustments while athletic or individual-based protocols may incorporate lean body mass calculations for enhanced precision. The system may implement these physiological correlations during both training data collection phases, where fluid intake protocols are adjusted to ensure representative PPG sampling across different body compositions, and during operational inference phases, where hydration recommendations are personalized based on the user's calculated blood volume and plasma volume characteristics to provide physiologically appropriate fluid intake guidance.

The individual-based approach creates personalized models through user-specific training protocols implemented via dedicated interface-sensor system 115 operation. The user interface 120 guides individual users through the complete hydration protocol, with the data manager 202B storing user-specific PPG and temperature data locally while the server interface manager 202C transmits this personalized dataset to the server 105 for individual model generation.

Individual model training captures user-specific physiological responses through extended operation of the PPG sensor 125 and other sensors 130. The temperature sensor integration provides personalized thermal response patterns during hydration changes, while the PPG sensor 125 records individual variations in cardiovascular response, skin properties affecting signal characteristics, and personal hydration kinetics. This comprehensive physiological profiling enables the model generator 302D to create highly personalized hydration assessment algorithms.

The individual training process may extend beyond the standard 1-liter population scale through adaptive protocol management by the routine manager 302C. For athletes and individuals in extreme conditions, the system implements the extended individual scale with 200-250 ml increments beyond 1 liter, potentially reaching 1.5-2 liters or higher. The UX manager 202D provides user guidance through these extended protocols, while plateau detection algorithms analyze PPG and temperature data to determine when the user's personal maximum hydration state has been achieved.

The two-tier system enables flexible deployment through coordinated operation of the platform model manager 302A and individual model managers 202A. New users receive immediate access to the population-based model through the device manager 302E distribution system, enabling hydration monitoring capability from initial device setup. The user interface 120 can simultaneously collect individual usage data through the data manager 202B, progressively building a personalized dataset for potential individual model training.

The system implements hybrid approaches where the population model provides baseline functionality while individual data progressively personalizes the model through continued use. The server interface manager 202C transmits individual usage patterns and hydration responses to the server 105, where the platform model manager 302A can identify users who would benefit from individual model training based on deviation patterns from population norms or specific accuracy requirements.

In some case, individual models represent calibrated versions of the population model, as implemented through the model generator 302D, where user-specific data refines the general model parameters rather than creating entirely separate algorithms. This approach leverages the statistical robustness of population training while incorporating personal physiological characteristics through transfer learning techniques that adapt the population model architecture to individual response patterns.

The individual-based model may operate through multiple implementation approaches that enable personalized hydration assessment while maintaining computational efficiency and accuracy. In some cases, the individual model may begin as a completely new model trained exclusively on user-specific data collected through personalized hydration protocols, generating algorithms that reflect the individual's unique physiological characteristics, cardiovascular responses, and hydration kinetics. Alternatively, the individual model may utilize transfer learning techniques where a pre-trained population-based model serves as the foundation, with individual user data progressively refining the model parameters through continued training sessions that adapt the general algorithms to personal physiological patterns. Following initial model establishment, the individual-based system may implement continuous learning capabilities that dynamically update dehydration reference points whenever new instances of dehydration are detected that exceed previously recorded levels. The system may automatically adjust the “dry” point of the hydration scale downward when more severe dehydration states are encountered, subsequently recalculating the entire hydration assessment scale based on these updated baseline values to maintain accurate relative hydration classifications. In some aspects, this adaptive scaling approach may enable the individual model to account for physiological changes over time, seasonal variations in baseline hydration, or evolving activity patterns that may shift the user's typical hydration range, ensuring that hydration assessments remain calibrated to the individual's current physiological state rather than static historical baselines.

Both training tiers benefit from the signal processing improvements implemented in the preprocess layer of the model architecture, including bandpass filtering to extract the 0.5-6 Hz frequency range and feature parameter reduction from 512 to 112 parameters. The temperature sensor integration through other sensors 130 provides additional input features for both population and individual model training, enhancing hydration state determination accuracy across all user categories.

The BMI correlation system operates at both training tiers through the platform data manager 302B, which adjusts training protocols based on user body composition data. Population model training incorporates BMI-adjusted fluid intake requirements to ensure representative training data across body composition ranges, while individual training personalizes fluid intake recommendations based on the user's specific BMI and physiological response patterns.

The routine manager 302C coordinates the implementation of both training approaches, managing population-wide data collection schedules while accommodating individual training requests. This coordinated approach ensures that all users receive the advantages of enhanced measurement and analysis capabilities, including multi-sensor data integration, optimized signal processing, and physiologically-informed training protocols, regardless of their chosen training approach.

Thus, the present disclosure provides a system for hydration monitoring and alerting that leverages photoplethysmogram data analysis and machine learning techniques to assess hydration status in real-time. The system comprises two primary components: a server and an interface-sensor system, which work in coordination to collect physiological data, process the data through trained models, and provide hydration state assessments to users. This distributed architecture enables both population-level model development and individualized hydration monitoring capabilities.

The server component may be configured to obtain a set of photoplethysmogram (PPG) data samples for a set of population users during controlled hydration protocols. The server marks each PPG data sample of the set of PPG data samples with a selected label selected from a set of labels, to thereby obtain a set of training data that correlates physiological measurements with known hydration states. The selected label indicates a hydration level of a population user of the set of population users at a specific time that corresponds to when a respective PPG data sample was obtained, establishing temporal relationships between PPG signal characteristics and hydration conditions. The set of labels includes at least two labels that indicate different hydration levels of the set of population users, enabling the system to distinguish between various hydration states. In some cases, the at least two labels include a first label for dehydration and a second label for hydration, providing a foundational binary classification framework for hydration assessment.

Using the labeled training data, the server may generate a model based on the set of training data through machine learning processes that identify patterns and relationships between PPG signal features and hydration states. The model development process may involve training neural networks, convolutional architectures, or other machine learning algorithms to recognize physiological signatures associated with different levels of hydration. The server may employ various preprocessing techniques, feature extraction methods, and model optimization approaches to enhance the accuracy and reliability of hydration state predictions. In some aspects, the model generation process may incorporate data from multiple sensor types, demographic information, and environmental factors to improve prediction capabilities across diverse user populations.

The interface-sensor system comprises a PPG sensor and a user interface that work together to collect real-time physiological data and provide hydration assessments to users. The PPG sensor may be configured to obtain a current PPG data sample for a user through optical measurement techniques that detect blood volume changes in peripheral tissues. The PPG sensor may utilize specific wavelengths of light to penetrate skin tissue and measure variations in light absorption that correspond to cardiovascular activity and blood flow characteristics. These measurements may be obtained continuously or at regular intervals, depending on the monitoring mode and user activity level.

The user interface may be configured to obtain the current PPG data sample from the PPG sensor, and indicate a hydration state of the user based on the current PPG data sample through a series of computational processes. The user interface obtains the model from the server or local storage, enabling real-time analysis of PPG data using the trained algorithms. The user interface processes the current PPG data sample through the model to obtain a hydration score for the user, which represents a quantitative assessment of the user's hydration level based on the physiological patterns detected in the PPG signal. Based on the hydration score of the user, the user interface determines a hydration state of the user by comparing the score to predetermined thresholds or classification boundaries established during model training.

Following hydration state determination, the user interface may output an indicator based on the hydration state to provide feedback to the user about their current hydration condition. The indicator may take various forms, including visual displays, auditory alerts, haptic feedback, or combinations of these output modalities. In some cases, the indicator may include specific recommendations for fluid intake, warnings about dehydration risk, or confirmations of adequate hydration status. The user interface may also provide historical data, trend analysis, and personalized insights based on the user's hydration patterns over time.

The system architecture enables continuous monitoring and assessment of hydration status through the integration of sensor data collection, machine learning analysis, and user feedback mechanisms. The distributed processing approach allows for both real-time local analysis and cloud-based model updates, ensuring that users receive immediate hydration assessments while benefiting from ongoing improvements to the underlying algorithms. In some aspects, the system may support multiple users simultaneously, with each user receiving personalized hydration monitoring based on their individual physiological characteristics and hydration patterns.

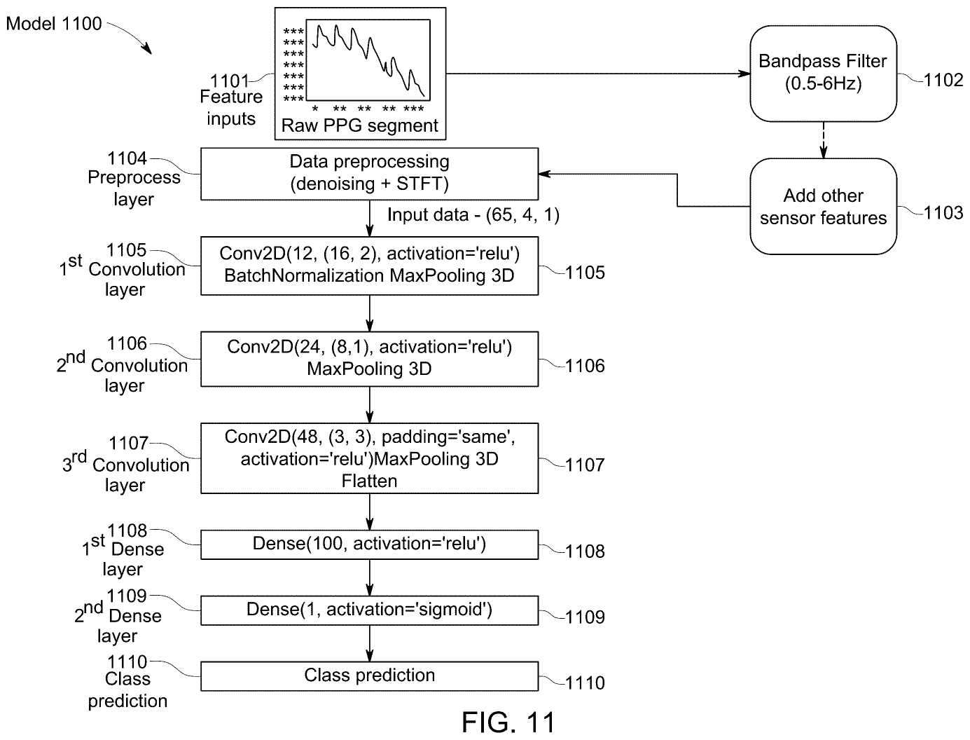

FIG. 11 depicts a model 1100 for processing photoplethysmogram data in the hydration monitoring system. The model 1100 represents an enhanced neural network architecture that incorporates advanced signal processing techniques and multi-sensor integration capabilities to improve hydration state determination accuracy. The model 1100 may be implemented within the server 105 during training phases and deployed to the user interface 120 for real-time hydration assessment. The architecture shown in FIG. 11 demonstrates the sequential processing pipeline that transforms raw physiological data into actionable hydration classifications through multiple computational stages.

The model 1100 begins with feature inputs 1101 that serve as the entry point for physiological data collection and initial parameter assembly. The feature inputs 1101 may encompass a comprehensive set of parameters derived from multiple data sources, including PPG signal characteristics, user demographic information, activity measurements, and sleep pattern data. In some cases, the feature inputs 1101 may initially comprise 512 different parameters that capture various aspects of the user's physiological state and contextual information. The feature inputs 1101 may include user demographic data such as age, gender, body mass index, and medical history that influence individual hydration patterns and physiological responses. The feature inputs 1101 may also incorporate user activity data including exercise intensity, duration, environmental conditions, and movement patterns that affect fluid loss rates and hydration requirements. Additionally, the feature inputs 1101 may encompass user sleep data such as sleep duration, sleep quality metrics, and circadian rhythm patterns that influence baseline hydration states and recovery processes.

Following the feature inputs 1101, the model 1100 incorporates a bandpass filter step 1102 that performs targeted frequency domain filtering to isolate physiologically relevant signal components. The bandpass filter step 1102 may operate on digitized analog signals that have been sampled at high frequencies, such as 36,000 times per second, to capture detailed temporal characteristics of the PPG waveform. The bandpass filter step 1102 may extract frequency components within a range of 0.5 to 6 Hz, which encompasses the physiological frequency spectrum associated with cardiovascular activity and hydration-related signal variations. In some cases, the bandpass filter step 1102 may eliminate low-frequency drift artifacts below 0.5 Hz that may result from sensor movement, ambient temperature fluctuations, or baseline signal drift that does not contribute meaningful physiological information for hydration assessment. The bandpass filter step 1102 may also remove high-frequency noise above 6 Hz that may originate from electrical interference, motion artifacts, or sensor noise that can degrade signal quality and reduce classification accuracy.

In some aspects, the bandpass filter may be configured to operate within the 0.5 to 6 Hz frequency range, though the system may accommodate various alternative frequency ranges depending on specific monitoring requirements and physiological conditions. The frequency range may be adjusted to be larger or smaller based on the target physiological signals and environmental conditions. For example, the lower frequency bound may be reduced to 0.1 Hz, 0.2 Hz, 0.3 Hz, or 0.4 Hz to capture slower physiological variations related to respiratory patterns or thermoregulatory responses, or may be extended down to 0.01 Hz to include very low frequency components associated with long-term physiological trends. The upper frequency bound may be increased to 8 Hz, 10 Hz, 15 Hz, 20 Hz, or up to 100 Hz to incorporate higher frequency cardiac components, motion artifacts for activity detection, or other rapid physiological changes that may correlate with hydration status. In some cases, the system may implement intermediate frequency ranges such as 0.3 to 8 Hz, 0.2 to 12 Hz, or 0.4 to 15 Hz to optimize signal capture for specific user populations or monitoring scenarios. The bandpass filter parameters may be dynamically adjusted based on signal quality assessment, user activity level, or environmental conditions, enabling adaptive frequency selection that maximizes the extraction of hydration-relevant physiological information while minimizing noise and artifact interference.

The model 1100 includes an add other sensor feature step 1103 that integrates additional physiological measurements to enhance hydration state determination capabilities. The add other sensor feature step 1103 may incorporate temperature measurements obtained using integrated sensors capable of measuring body temperature in real-time during various activity levels and environmental conditions. In some cases, the add other sensor feature step 1103 may process temperature data that correlates core temperature rises during exercise lasting multiple hours with progressive dehydration through perspiration and respiratory water loss mechanisms. The add other sensor feature step 1103 may synchronize temperature measurements with PPG data collection timing to ensure temporal alignment between different sensor modalities. The model architecture may include synchronized multi-parameter data collection where temperature measurements are obtained continuously or at regular intervals corresponding to PPG data collection timing, enabling correlation of temperature trends with PPG signal changes. The add other sensor feature step 1103 may also incorporate data from additional sensors such as skin conductance monitors, accelerometers, or environmental sensors that provide contextual information about the user's physiological state and surrounding conditions.

As shown in FIG. 11, the model 1100 incorporates a preprocess layer 1104 that performs comprehensive data conditioning and feature optimization operations on the combined sensor data. The preprocess layer 1104 may apply the bandpass filtering (discussed above) and feature parameter reduction to streamline the computational pipeline while maintaining prediction accuracy. In some cases, the preprocess layer 1104 may reduce the feature set from an initial collection of 512 parameters to a refined set of 112 parameters by eliminating redundant or noise-contributing features that provide minimal value to hydration state determination. The preprocess layer 1104 may employ statistical analysis, correlation assessment, and feature importance ranking to identify the most discriminative parameters for hydration classification. The preprocess layer 1104 may also perform data normalization, scaling, and transformation operations to ensure that different sensor modalities and parameter types are appropriately weighted and formatted for subsequent neural network processing.

In some aspects, the feature parameter reduction process may be adapted based on deployment requirements, sensor configurations, and user characteristics, with the system capable of operating across a range of parameter sets from the initial 512 parameters to various refined configurations. The reduction to 112 parameters represents one optimization approach, though the system may implement alternative parameter counts such as 64, 96, 128, 156, 200, or 256 parameters depending on computational constraints, battery life requirements, and accuracy targets for specific applications. The parameter selection process may vary based on the available sensor types, where systems incorporating only PPG sensors may utilize fewer parameters than multi-modal implementations that include temperature sensors, accelerometers, or environmental monitoring devices. In some cases, user-specific factors such as age, activity level, medical conditions, or physiological characteristics may influence the optimal parameter set, with the system dynamically selecting feature subsets that provide maximum discriminative value for individual hydration assessment. The feature elimination process may prioritize removal of parameters that contribute primarily noise or redundancy for specific deployment scenarios, such as eliminating motion-related features for sedentary monitoring applications or reducing spectral parameters in environments with consistent ambient conditions. The system may implement adaptive feature selection algorithms that continuously evaluate parameter relevance based on signal quality, user feedback, and prediction accuracy, enabling real-time optimization of the feature set to maintain optimal hydration state determination performance while minimizing computational overhead and power consumption.

The model 1100 includes a series of convolutional layers that extract spatial and temporal features from the preprocessed physiological data. A first convolution layer 1105 may apply initial feature extraction operations using convolutional filters designed to detect local patterns and relationships within the PPG signal and associated sensor data. The first convolution layer 1105 may be configured to extract spatial features from preprocessed PPG data through convolution operations that identify characteristic waveform shapes, amplitude variations, and temporal patterns associated with different hydration states. Following the first convolution layer 1105, a second convolution layer 1106 may perform more complex feature extraction operations that build upon the patterns identified in the previous layer. The second convolution layer 1106 may apply additional convolutional filters with different kernel sizes and parameters to capture higher-level features and relationships within the physiological data. A third convolution layer 1107 may complete the convolutional processing pipeline by extracting the most abstract and discriminative features that represent complex physiological patterns associated with hydration states.

With continued reference to FIG. 11, the model 1100 incorporates dense layers that perform high-level feature integration and classification preparation. A first dense layer 1108 may process the output from the convolutional layers through fully connected neural network operations that combine and weight the extracted features according to their relevance for hydration state determination. The first dense layer 1108 may be configured to process temperature data as additional input parameters alongside PPG-derived features, enabling the model to learn complex relationships between temperature variations and PPG signal characteristics. In some cases, the first dense layer 1108 may identify dehydration states more accurately than PPG data alone by incorporating thermal information that reflects physiological stress and fluid balance changes. A second dense layer 1109 may perform final feature processing and dimensionality reduction to prepare the integrated feature representation for classification operations. The second dense layer 1109 may apply activation functions, regularization techniques, and other neural network operations to optimize the feature representation for accurate hydration state prediction.

The model 1100 concludes with a class prediction module 1110 that generates the final hydration state classification based on the processed physiological features. The class prediction module 1110 may apply classification algorithms such as softmax activation functions, sigmoid operations, or other decision-making mechanisms to convert the processed features into discrete hydration state categories or continuous hydration scores. In some cases, the class prediction module 1110 may output probability distributions across multiple hydration states, enabling the system to provide confidence measures and uncertainty estimates along with the primary classification result. The class prediction module 1110 may be configured to learn complex relationships between temperature variations and PPG signal characteristics through the training process, enabling the model to identify subtle physiological patterns that indicate hydration state changes. The model 1100 may implement fine-tuning processes where individual data progressively personalizes general model parameters while leveraging statistical robustness of population training, allowing the system to adapt to individual user characteristics while maintaining broad applicability across diverse user populations.

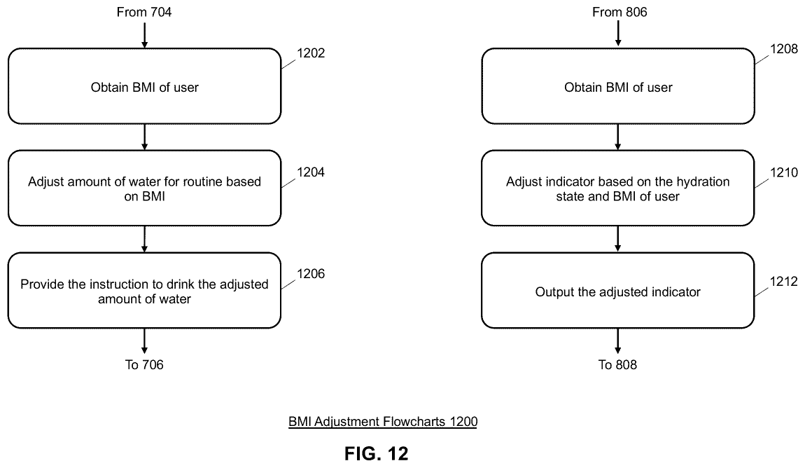

FIG. 12 depicts BMI adjustment flowcharts 1200 that illustrate the systematic processes for incorporating Body Mass Index correlations into hydration monitoring protocols. The BMI adjustment flowcharts 1200 demonstrate how the hydration monitoring system may adjust fluid intake recommendations and hydration state indicators based on individual user BMI measurements during both training data collection phases and operational monitoring periods. The BMI adjustment flowcharts 1200 may be implemented within the server 105 and user interface 120 to ensure that hydration assessments account for physiological differences in blood volume and fluid requirements across users with varying body compositions. The flowcharts shown in FIG. 12 represent different processing pathways that may operate independently to provide BMI-adjusted recommendations during routine execution and BMI-adjusted indicators during hydration state determination.

The left flowchart in the BMI adjustment flowcharts 1200 begins with a step 1202 that obtains BMI of user from step 704 of FIG. 7, establishing the user's body mass index as a foundational parameter for fluid intake calculations. The step 1202 may retrieve BMI data from user profile information, calculate BMI from height and weight measurements, or obtain BMI values through direct user input or connected health monitoring devices. Following BMI acquisition, the process advances to a step 1204 that adjusts amount of water for routine based on BMI, implementing mathematical correlations that account for physiological differences in blood volume and fluid requirements. The step 1204 may apply specific mathematical correlations for individuals based on BMI to adjust fluid intake compared to normal-weight individuals, reflecting the increased blood volume and metabolic demands associated with higher body mass. The adjustment process concludes with a step 1206 that provides the instruction to drink the adjusted amount of water at step 706 of FIG. 7, ensuring that hydration protocols are tailored to individual physiological characteristics rather than applying uniform fluid intake recommendations across all users.

With continued reference to FIG. 12, the right flowchart in the BMI adjustment flowcharts 1200 demonstrates the process for adjusting hydration state indicators based on user BMI during operational monitoring phases. The process initiates with a step 1208 that obtains BMI of user from step 804 of FIG. 8, retrieving the same BMI parameters used in the training adjustment process to maintain consistency across system operations. The step 1208 may access stored BMI values from user profiles or recalculate BMI based on updated user measurements to ensure that indicator adjustments reflect current body composition characteristics. The process continues with a step 1210 that adjusts indicator based on the hydration state and BMI of user, modifying the hydration assessment output to account for BMI-specific physiological differences in fluid balance and hydration requirements. The step 1210 may implement algorithms that scale hydration indicators according to the user's BMI category, ensuring that dehydration warnings and hydration confirmations are appropriately calibrated for individuals with different body compositions. The adjustment process concludes with a step 1212 that outputs the adjusted indicator at step 806 of FIG. 8, providing users with BMI-corrected hydration feedback that reflects their individual physiological characteristics and fluid balance requirements.

The BMI adjustment flowcharts 1200 may implement physiological correlations based on blood volume scaling with body mass, with circulating blood volumes ranging from approximately 4.9 liters in normal-weight individuals to 7 liters in individuals with elevated BMI. The mathematical relationships encoded in the BMI adjustment flowcharts 1200 may account for the fact that adipose tissue requires vascular supply for nutrient delivery and waste removal, resulting in proportionally larger blood volumes as BMI increases. In some cases, the BMI adjustment flowcharts 1200 may calculate fluid intake adjustments using linear or non-linear scaling functions that correlate BMI values with blood volume estimates, ensuring that hydration recommendations reflect the physiological reality of increased fluid requirements for individuals with higher body mass. The server 105 or the user interface 120 may adjust fluid intake recommendations during training data collection or adjust fluid intake during routines based on user BMI to ensure data accurately reflects the relationship between PPG signals and hydration states across different body compositions.

As shown in FIG. 12, the BMI adjustment flowcharts 1200 may incorporate feedback mechanisms that allow the system to refine BMI-based adjustments over time based on user response patterns and physiological data collected during hydration monitoring sessions. The step 1204 and step 1210 may access historical data about user hydration responses to BMI-adjusted recommendations, enabling the system to calibrate adjustment algorithms based on individual user characteristics and population-level trends. In some cases, the BMI adjustment flowcharts 1200 may implement machine learning algorithms that analyze the relationship between BMI adjustments, fluid intake compliance, and hydration state outcomes to optimize the mathematical correlations used in the adjustment processes. The BMI adjustment flowcharts 1200 may also incorporate safety limits and validation checks to ensure that BMI-based adjustments remain within physiologically reasonable ranges and do not recommend excessive fluid intake that could lead to overhydration or other adverse effects.

The implementation of the BMI adjustment flowcharts 1200 may enable the hydration monitoring system to provide personalized hydration guidance that accounts for individual physiological differences while maintaining the statistical robustness of population-based model training. The step 1202 and step 1208 may retrieve BMI data from the same user profile sources to ensure consistency between training and operational phases, while the step 1204 and step 1210 may apply corresponding adjustment algorithms that maintain proportional relationships between training protocols and real-time monitoring feedback. The BMI adjustment flowcharts 1200 may support the model 1100 by ensuring that training data collected through BMI-adjusted protocols accurately represents the physiological responses of users across different body composition categories, enabling the feature inputs 1101 to incorporate BMI-corrected parameters that enhance the accuracy of hydration state predictions generated by the class prediction module 1110.

### 9. Graphical User Interfaces

The hydration monitoring system incorporates comprehensive user interface sequences that facilitate user interaction, data collection, and hydration assessment across multiple operational modes. The interface sequences may be implemented through the user interface 120 and may support both population-based and individual training approaches through various interaction pathways. The user interface sequences may provide structured workflows that guide users through system setup, profile configuration, real-time monitoring, data logging, analytics review, and system configuration processes. In some cases, the interface sequences may integrate with the model 1100 to provide real-time hydration assessments and may incorporate data from the BMI adjustment flowcharts 1200 to personalize user experiences based on individual physiological characteristics.

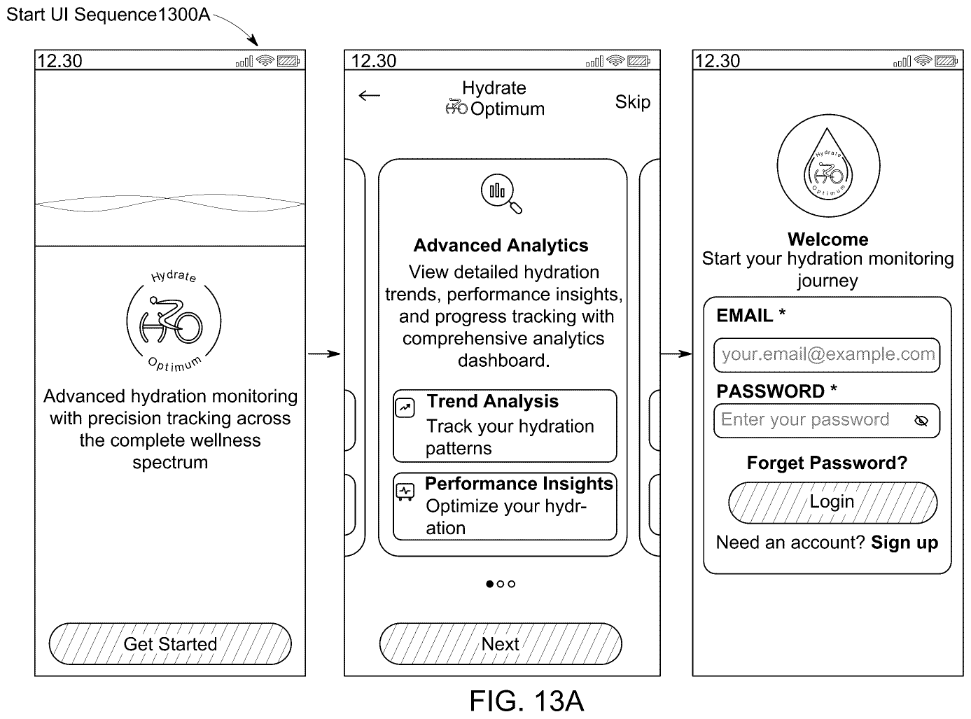

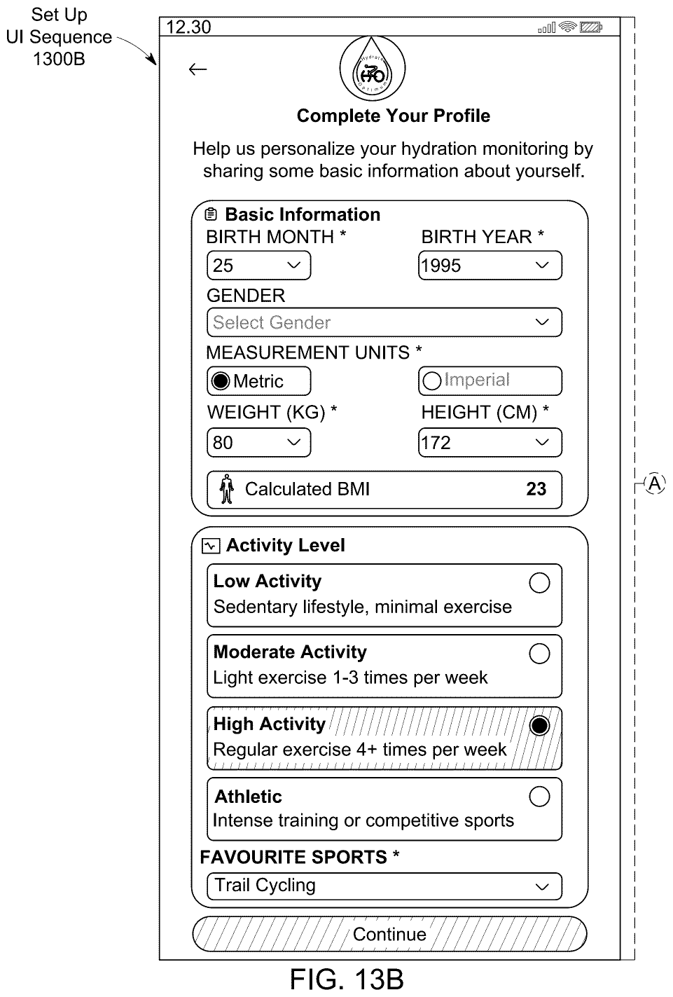

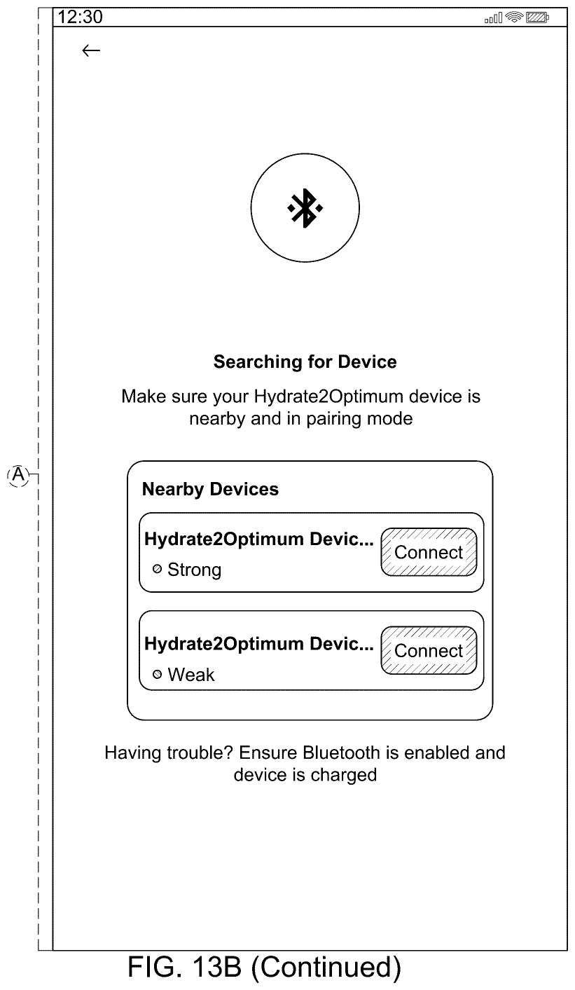

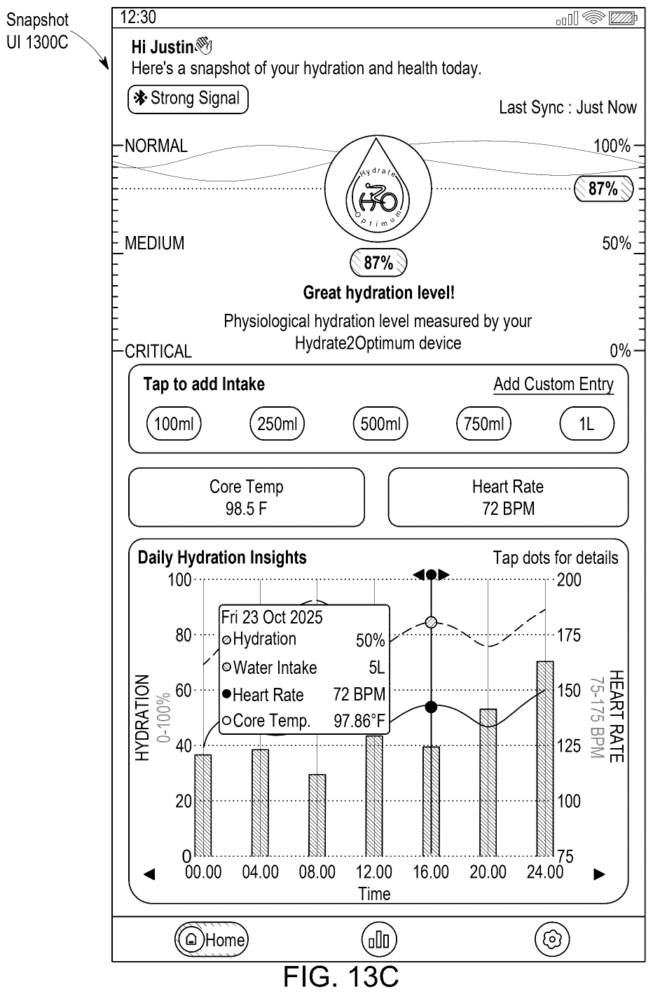

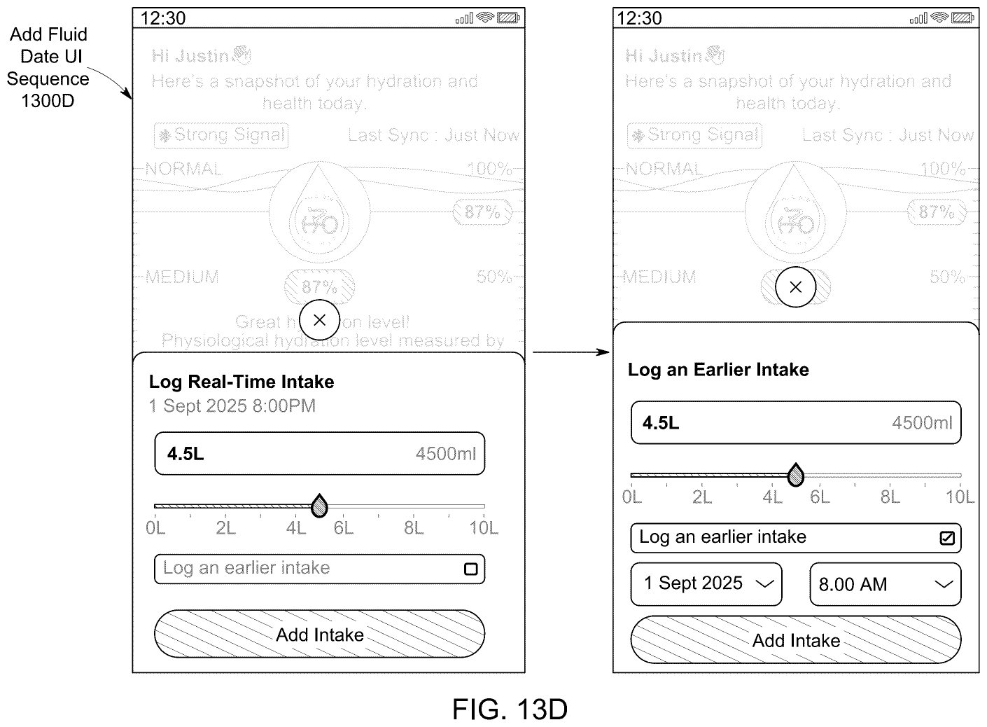

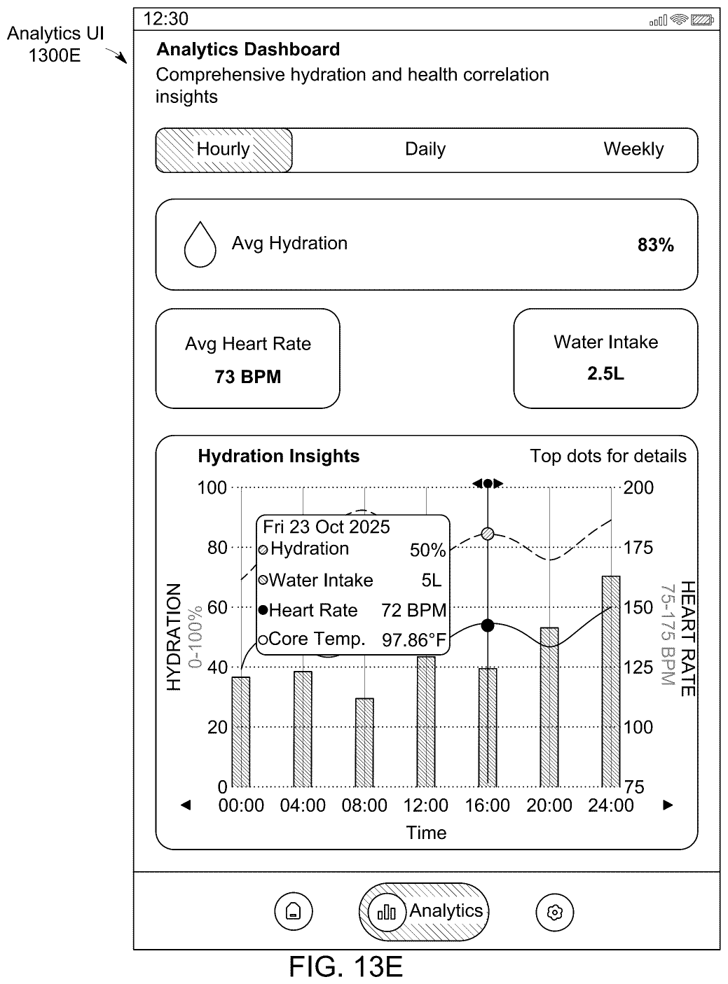

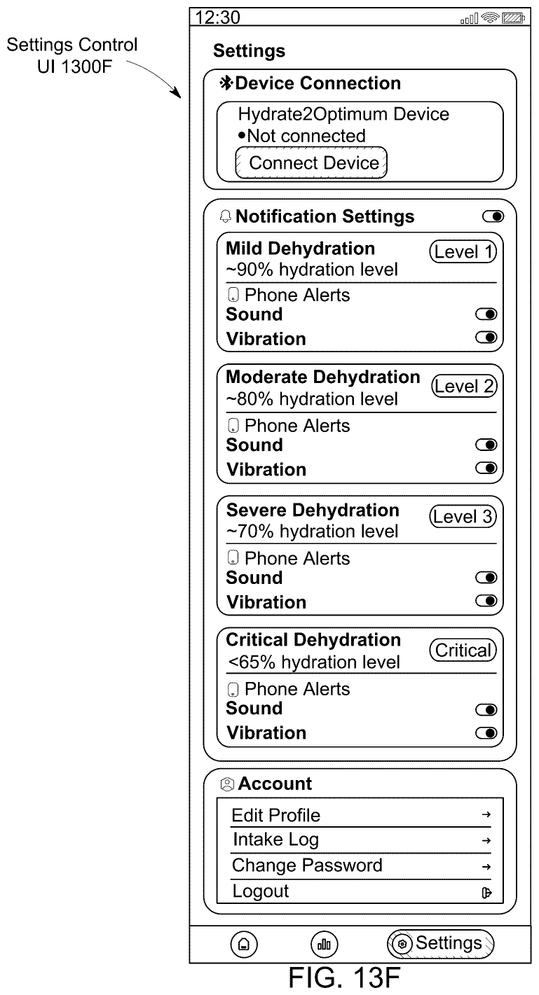

FIG. 13A depicts a start UI sequence 1300A that provides the initial user interaction pathway for accessing the hydration monitoring system. The start UI sequence 1300A may present users with introductory information about the system capabilities, advanced hydration monitoring features, and authentication mechanisms for accessing personalized hydration services. The start UI sequence 1300A may include welcome screens that communicate the system's advanced analytics capabilities, trend analysis features, and performance insights that users may access through the hydration monitoring platform. In some cases, the start UI sequence 1300A may incorporate login interfaces with email and password input fields, along with options for password recovery and new account creation to ensure secure access to personalized hydration data. The start UI sequence 1300A may serve as the entry point for both new users beginning their hydration monitoring journey and returning users accessing their established hydration profiles and historical data.

With continued reference to FIG. 13A, the start UI sequence 1300A may implement progressive disclosure techniques that introduce system features and capabilities in a structured manner to avoid overwhelming new users with complex functionality. The start UI sequence 1300A may present advanced analytics features including trend analysis and performance insights options that connect to the analytics capabilities supported by the model 1100 and the class prediction module 1110. The authentication components within the start UI sequence 1300A may establish secure connections to the server 105 to retrieve user profiles, historical hydration data, and personalized model parameters that have been developed through previous training sessions. In some cases, the start UI sequence 1300A may include onboarding elements that explain the two-tier training approach, allowing users to understand the differences between population-based and individual training methodologies before proceeding with system setup.

FIG. 13B illustrates a set up UI sequence 1300B that guides users through profile configuration and device connection processes that establish the foundation for personalized hydration monitoring. The set up UI sequence 1300B may include profile completion interfaces that collect user demographic information, physiological parameters, and activity preferences that serve as inputs to the feature inputs 1101 of the model 1100. The set up UI sequence 1300B may incorporate BMI calculation functionality that automatically computes body mass index values based on user-provided height and weight measurements, enabling the system to apply the BMI adjustment flowcharts 1200 for personalized fluid intake recommendations. In some cases, the set up UI sequence 1300B may include activity level selection options ranging from low activity to athletic categories, along with favorite sports dropdown menus that help the system understand user activity patterns and hydration requirements.

As shown in FIG. 13B, the set up UI sequence 1300B may include device connection interfaces that facilitate pairing between the user interface 120 and the PPG sensor 125 through wireless communication protocols. The set up UI sequence 1300B may present device searching interfaces that identify nearby hydration monitoring devices and display connection strength indicators to help users establish reliable sensor connections. The device connection process within the set up UI sequence 1300B may include pairing mode instructions and connection status feedback that ensures proper communication between the interface-sensor system components. In some cases, the set up UI sequence 1300B may incorporate measurement unit selection options with radio buttons for metric and imperial units, allowing users to configure the system according to their preferred measurement standards for weight, height, and fluid volume displays.

FIG. 13C depicts a snapshot UI 1300C that provides real-time hydration monitoring displays and immediate user interaction capabilities during active monitoring sessions. The snapshot UI 1300C may present hydration level indicators that display current hydration percentages prominently, along with visual elements such as water droplet icons that provide intuitive representations of hydration status. The snapshot UI 1300C may incorporate fluid intake buttons that offer quick logging options for common fluid volumes, such as 100 ml, 250 ml, 500 ml, 750 ml, and 1 L, enabling users to record fluid consumption without complex data entry procedures. In some cases, the snapshot UI 1300C may display core temperature readings and heart rate measurements that correspond to data collected through the add other sensor feature step 1103, providing users with comprehensive physiological feedback beyond hydration status alone.

With continued reference to FIG. 13C, the snapshot UI 1300C may include daily hydration insights graphs that visualize multiple physiological parameters over time, including hydration levels, water intake volumes, heart rate measurements, and core temperature readings. The snapshot UI 1300C may process real-time data through the model 1100 to generate current hydration assessments that are displayed through the hydration level indicators and status messages. The temperature and heart rate displays within the snapshot UI 1300C may reflect data processing through the first dense layer 1108 and second dense layer 1109, which integrate multiple sensor inputs to provide comprehensive physiological assessments. In some cases, the snapshot UI 1300C may incorporate navigation elements that allow users to access different system functions while maintaining awareness of their current hydration status through persistent display elements.

FIG. 13D illustrates an add fluid data UI sequence 1300D that enables users to log fluid intake information through structured data entry workflows that support both real-time and retrospective data recording. The add fluid data UI sequence 1300D may include fluid volume sliders that allow users to specify precise intake amounts, along with timestamp controls that enable accurate temporal recording of fluid consumption events. The add fluid data UI sequence 1300D may support the training data collection processes described in the BMI adjustment flowcharts 1200 by providing mechanisms for users to record fluid intake during controlled hydration sessions. In some cases, the add fluid data UI sequence 1300D may include date and time selection fields that allow users to log fluid intake events that occurred at different times, supporting retrospective data entry for users who may not have immediate access to the system during fluid consumption.

As shown in FIG. 13D, the add fluid data UI sequence 1300D may incorporate validation mechanisms that ensure data accuracy and consistency with physiological constraints and system expectations. The add fluid data UI sequence 1300D may provide visual feedback through fluid volume displays that confirm user selections before data submission, reducing the likelihood of data entry errors that could affect model training or hydration assessment accuracy. The temporal controls within the add fluid data UI sequence 1300D may support the incremental hydration protocols that are processed through the preprocess layer 1104 and subsequent neural network layers to establish personalized hydration baselines. In some cases, the add fluid data UI sequence 1300D may integrate with the server 105 to transmit fluid intake data for population-based model training or individual model calibration processes.

FIG. 13E depicts an analytics dashboard UI 1300E that provides comprehensive data visualization and trend analysis capabilities for users to review their hydration patterns and physiological responses over extended time periods. The analytics dashboard UI 1300E may include tabbed navigation interfaces with hourly, daily, and weekly view options that allow users to examine their hydration data at different temporal resolutions. The analytics dashboard UI 1300E may display average hydration levels, heart rate measurements, and water intake volumes that are calculated from data processed through the model 1100 and stored through the data collection workflows. In some cases, the analytics dashboard UI 1300E may incorporate hydration insights graphs that visualize correlations between multiple physiological parameters, enabling users to understand relationships between hydration status, cardiovascular activity, temperature variations, and fluid intake patterns.

With continued reference to FIG. 13E, the analytics dashboard UI 1300E may implement data processing algorithms that analyze historical information collected through the feature inputs 1101 and processed through the bandpass filter step 1102 to identify long-term trends and patterns in user hydration behavior. The analytics dashboard UI 1300E may provide statistical summaries and trend indicators that help users understand their hydration patterns relative to their activity levels, environmental conditions, and physiological responses. The multi-parameter visualization capabilities within the analytics dashboard UI 1300E may reflect the integrated sensor processing performed through the add other sensor feature step 1103, presenting temperature, heart rate, and hydration data in coordinated displays that reveal physiological relationships. In some cases, the analytics dashboard UI 1300E may include comparison features that allow users to evaluate their current hydration performance against historical baselines or population-level benchmarks derived from the population-based model training processes.

FIG. 13F illustrates a settings control UI 1300F that provides system configuration options and user account management capabilities that enable personalization of the hydration monitoring experience. The settings control UI 1300F may include device connection sections that display current sensor connectivity status and provide options for establishing or modifying connections between the user interface 120 and associated physiological monitoring devices. The settings control UI 1300F may incorporate notification settings sections that allow users to configure hydration level alerts for different dehydration stages, including mild, moderate, severe, and critical dehydration levels that correspond to classification outputs from the class prediction module 1110. In some cases, the settings control UI 1300F may provide sound and vibration alert toggles that enable users to customize notification modalities according to their preferences and usage contexts.

As shown in FIG. 13F, the settings control UI 1300F may include account management sections that provide access to profile editing, data logging review, password modification, and logout functionality that maintains user control over their personal information and system access. The settings control UI 1300F may integrate with the BMI adjustment flowcharts 1200 through profile editing options that allow users to update height, weight, and activity level information that affects personalized fluid intake recommendations and hydration assessment calibration. The notification configuration options within the settings control UI 1300F may correspond to the hydration state classifications generated through the first convolution layer 1105, second convolution layer 1106, and third convolution layer 1107 processing pathways, enabling users to receive alerts that are tailored to their individual hydration patterns and risk profiles. In some cases, the settings control UI 1300F may provide data export options, privacy controls, and system diagnostic information that support user autonomy and system transparency in the hydration monitoring process.

The hydration monitoring system may implement a comprehensive two-tier training approach that balances broad applicability with personalized accuracy through distinct methodological pathways. The two-tier approach may comprise a population-based model trained on PPG data from multiple subjects across diverse demographic and physiological characteristics, and an individual-based model created through user-specific training protocols for enhanced accuracy. The population-based model may provide immediate deployment capability for new users without requiring extensive individual calibration periods, while the individual-based model may offer enhanced precision for applications requiring detailed hydration management. The server may be configured to manage both training approaches simultaneously, enabling users to transition between methodologies based on their hydration monitoring requirements and commitment to personalized calibration processes.

The population-based training methodology may utilize data collection from multiple users across diverse demographic and physiological characteristics to develop robust models that generalize across user populations. The population-based approach may incorporate PPG data samples collected during standardized hydration protocols that account for variations in age, gender, body composition, activity levels, and environmental conditions. The server may aggregate anonymized data from multiple users to identify common physiological patterns associated with different hydration states, enabling the development of models that perform reliably across diverse user populations. In some cases, the population-based training may incorporate statistical techniques that weight data contributions based on demographic representation, ensuring that the resulting models provide accurate predictions for users across different population segments. The population-based model may serve as a foundation for immediate hydration monitoring capability, allowing new users to receive hydration assessments without completing individual calibration protocols.

New users may begin with the population-based model for immediate hydration monitoring capability and optionally upgrade to individual-based training for enhanced precision based on their specific requirements and usage patterns. The transition from population-based to individual-based training may occur gradually, with the system collecting user-specific data during normal operation to progressively personalize the model parameters. The model may support hybrid deployment strategies where users begin with population-based models and progressively personalize through continued individual data collection, enabling seamless enhancement of prediction accuracy over time. The hybrid approach may allow users to receive immediate hydration feedback while the system accumulates sufficient individual data to support personalized model calibration. In some cases, the system may automatically recommend individual training protocols when user data patterns suggest that personalized calibration would provide substantial accuracy improvements over the population-based approach.

The individual-based training approach may create personalized models through user-specific protocols that capture individual physiological characteristics and hydration response patterns. The individual-based model may involve the user performing a complete hydration protocol personally, generating PPG data specific to their physiological responses including cardiovascular response variations, skin properties, and personal hydration kinetics. The individual training process may extend beyond a standard 1-liter population scale and continue until plateau detection indicates the user's personal maximum hydration state has been achieved. The individual-based approach may account for personal variations in cardiovascular response to hydration changes, skin optical properties that affect PPG signal characteristics, and individual hydration kinetics (e.g., BMI, BV, muscle mass, or Hb/Hct concentration changes) that influence the temporal relationship between fluid intake and physiological response. In some cases, the individual training may involve multiple sessions conducted over several days to establish comprehensive baseline measurements and response patterns that reflect the user's unique physiological characteristics.

The user interface may be configured to implement a non-linear hydration scale for training data collection that reflects physiological realities of fluid loss and replacement patterns across different user populations. The non-linear hydration scale may include fluid volume increments of 0 ml. 500 ml, 700 ml, 800 ml, 900 ml, and 1000 ml that correspond to statistically probable dehydration levels in general populations. The non-linear hydration scale may provide finer resolution at higher dehydration levels to capture physiological changes that occur during moderate to severe fluid deficits. The scale design may provide 100 ml increments between 700 ml and 1000 ml to reflect higher probability of moderate fluid loss compared to extreme dehydration states. The non-linear progression may acknowledge that moderate dehydration states representing 100-200 ml additional fluid loss occur more frequently than severe dehydration, requiring more precise measurement and classification capabilities for accurate hydration assessment.

For individual-based training targeting athletes, the hydration scale may extend beyond 1000 ml with additional increments of 200-250 ml, potentially reaching 1.5 liters or higher to accommodate the increased fluid requirements associated with endurance activities and extreme environmental conditions. The extended scale for athletic applications may recognize that individuals engaged in prolonged physical activity may experience fluid losses that exceed typical population norms, necessitating broader measurement ranges for accurate hydration assessment. The athletic scale may maintain the principle of smaller increments at higher dehydration levels while extending the total range to capture physiological states that occur during extended exercise periods, high-temperature environments, or other conditions that promote substantial fluid loss. In some cases, the individual training process for athletes may continue beyond the standard population scale until plateau detection mechanisms indicate that the user's personal maximum hydration state has been achieved, regardless of the total fluid volume required to reach that state.

The system may be configured to detect plateau states during individual training by analyzing PPG data using statistical measures including moving average stabilization, standard deviation reduction, and slope analysis approaching zero. The plateau detection algorithms may monitor PPG signal characteristics over time to identify when additional fluid intake no longer produces measurable changes in physiological parameters, indicating that maximum hydration has been reached. The moving average stabilization analysis may calculate running averages of PPG signal amplitude over specified time windows, detecting plateau conditions when the moving average remains within predetermined tolerance ranges for sustained periods. The standard deviation reduction analysis may monitor signal variability over time, identifying plateau states when signal variance decreases below threshold values that indicate physiological stabilization. The slope analysis may perform linear regression on recent PPG data points to calculate signal trends, detecting plateau conditions when regression slopes approach zero within statistical confidence intervals.

Plateau detection may involve comparing current PPG signals to previous hydration session data using correlation coefficients, relative change analysis, and statistical distance measures to determine when maximum/optimal hydration has been reached. The correlation coefficient analysis may calculate statistical relationships between current PPG signals and signals recorded during previous hydration sessions at similar time points, identifying plateau conditions when correlation values exceed predetermined thresholds that indicate physiological similarity. The relative change analysis may compute percentage changes in PPG signal characteristics compared to previous session data, detecting plateau states when relative changes fall below threshold values that indicate minimal physiological response to additional fluid intake. The statistical distance measures may calculate mathematical distances between current PPG signal distributions and distributions observed during previous plateau states, using techniques such as Kullback-Leibler divergence or Jensen-Shannon divergence to quantify physiological similarity. In some cases, the plateau detection system may combine multiple analytical approaches, weighting each method based on historical accuracy and individual user response patterns to enhance detection reliability and account for day-to-day physiological variations.

The training methodologies may incorporate adaptive protocols that adjust data collection procedures based on individual user characteristics, environmental conditions, and physiological response patterns observed during initial training sessions. The adaptive protocols may modify fluid intake increments, session timing, and data collection frequency based on user-specific factors such as body mass index, activity level, and baseline hydration patterns. The system may implement machine learning algorithms that analyze user response patterns during training to optimize subsequent session parameters, reducing the total time required to establish personalized hydration baselines while maintaining data quality and model accuracy. In some cases, the adaptive training protocols may incorporate environmental sensors and activity monitoring to account for external factors that influence hydration requirements and physiological responses during training sessions. The training methodologies may also include validation procedures that assess model accuracy using cross-validation techniques, holdout datasets, and real-world testing scenarios to ensure that both population-based and individual-based models provide reliable hydration assessments across diverse usage conditions and user populations.

### 10. Computer System

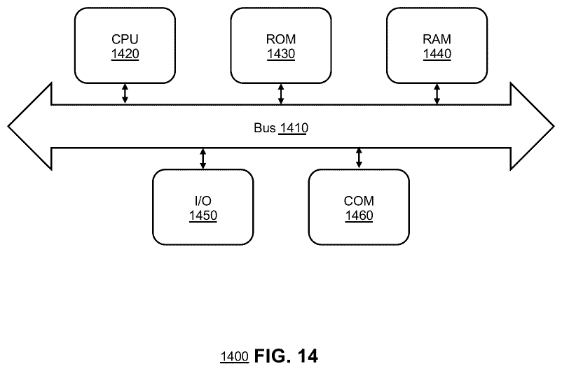

FIG. 14 depicts an example system that may execute techniques presented herein. FIG. 14 is a simplified functional block diagram of a computer that may be configured to execute techniques described herein, according to exemplary cases of the present disclosure. Specifically, the computer (or “platform” as it may not be a single physical computer infrastructure) may include a data communication interface 1460 for packet data communication. The platform may also include a central processing unit (“CPU”) 1420, in the form of one or more processors, for executing program instructions. The platform may include an internal communication bus 1410, and the platform may also include a program storage and/or a data storage for various data files to be processed and/or communicated by the platform such as ROM 1430 and RAM 1440, although the system 1400 may receive programming and data via network communications. The system 1400 also may include input and output ports 1450 to connect with input and output devices such as keyboards, mice, touchscreens, monitors, displays, etc. Of course, the various system functions may be implemented in a distributed fashion on a number of similar platforms, to distribute the processing load. Alternatively, the systems may be implemented by appropriate programming of one computer hardware platform.

The general discussion of this disclosure provides a brief, general description of a suitable computing environment in which the present disclosure may be implemented. In some cases, any of the disclosed systems, methods, and/or graphical user interfaces may be executed by or implemented by a computing system consistent with or similar to that depicted and/or explained in this disclosure. Although not required, aspects of the present disclosure are described in the context of computer-executable instructions, such as routines executed by a data processing device, e.g., a server computer, wireless device, and/or personal computer. Those skilled in the relevant art will appreciate that aspects of the present disclosure can be practiced with other communications, data processing, or computer system configurations, including: Internet appliances, hand-held devices (including personal digital assistants (“PDAs”)), wearable computers, all manner of cellular or mobile phones (including Voice over IP (“VoIP”) phones), dumb terminals, media players, gaming devices, virtual reality devices, multi-processor systems, microprocessor-based or programmable consumer electronics, set-top boxes, network PCs, mini-computers, mainframe computers, and the like. Indeed, the terms “computer,” “server,” and the like, are generally used interchangeably herein, and refer to any of the above devices and systems, as well as any data processor.

Aspects of the present disclosure may be embodied in a special purpose computer and/or data processor that is specifically programmed, configured, and/or constructed to perform one or more of the computer-executable instructions explained in detail herein. While aspects of the present disclosure, such as certain functions, are described as being performed exclusively on a single device, the present disclosure may also be practiced in distributed environments where functions or modules are shared among disparate processing devices, which are linked through a communications network, such as a Local Area Network (“LAN”), Wide Area Network (“WAN”), and/or the Internet. Similarly, techniques presented herein as involving multiple devices may be implemented in a single device. In a distributed computing environment, program modules may be located in both local and/or remote memory storage devices.

Aspects of the present disclosure may be stored and/or distributed on non-transitory computer-readable media, including magnetically or optically readable computer discs, hard-wired or preprogrammed chips (e.g., EEPROM semiconductor chips), nanotechnology memory, biological memory, or other data storage media. Alternatively, computer implemented instructions, data structures, screen displays, and other data under aspects of the present disclosure may be distributed over the Internet and/or over other networks (including wireless networks), on a propagated signal on a propagation medium (e.g., an electromagnetic wave(s), a sound wave, etc.) over a period of time, and/or they may be provided on any analog or digital network (packet switched, circuit switched, or other scheme).

Program aspects of the technology may be thought of as “products” or “articles of manufacture” typically in the form of executable code and/or associated data that is carried on or embodied in a type of machine-readable medium. “Storage” type media include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer of the mobile communication network into the computer platform of a server and/or from a server to the mobile device. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

### 11. Terminology

The terminology used above may be interpreted in its broadest reasonable manner, even though it is being used in conjunction with a detailed description of certain specific examples of the present disclosure. Indeed, certain terms may even be emphasized above; however, any terminology intended to be interpreted in any restricted manner will be overtly and specifically defined as such in this Detailed Description section. Both the foregoing general description and the detailed description are exemplary and explanatory only and are not restrictive of the features, as claimed.

As used herein, the terms “comprises,” “comprising,” “having,” including,” or other variations thereof, are intended to cover a non-exclusive inclusion such that a process, method, article, or apparatus that comprises a list of elements does not include only those elements, but may include other elements not expressly listed or inherent to such a process, method, article, or apparatus.

In this disclosure, relative terms, such as, for example, “about,” “substantially,” “generally,” and “approximately” are used to indicate a possible variation of +10% in a stated value.

As used herein, the terms “transmit,” “provide,” “receive,” and “obtain” may refer to the transfer or communication of data, information, or signals between various components or entities. This may include, but is not limited to, transmission over a network (such as a local area network, wide area network, or the Internet), transfer between devices (such as between computers, smartphones, or other electronic devices), communication between central processing units (CPUs) or graphics processing units (GPUs), exchange of information between microservices, transfer of data between software components within an environment, or any other form of data transfer or communication as indicated by the context in which the terms are used. The specific mode or medium of transmission or provision may vary depending on the particular implementation and system architecture.

As used herein, the term “module” may refer to software code, a software component, a software function, a software application, and firmware. As indicated by context, “module” may be logical, digital, analog, optical, electronic, or quantum implementations of operations or functions. A module may be implemented as a standalone unit or as part of a larger system. In some cases, a module may interact with other modules or components to perform specific tasks or operations within the system. As indicated by context or based on design preference, any two modules may be combined. As indicated by context or based on design preference, any module may be broken into two or more modules that provide some or all of the operations or functions of the single module. The specific implementation of module(s) may vary depending on the requirements of the system and the particular application.

The term “exemplary” is used in the sense of “example” rather than “ideal.” As used herein, the singular forms “a,” “an,” and “the” include plural reference unless the context dictates otherwise.

### 12. Examples

Exemplary embodiments of the systems and methods disclosed herein are described in the numbered paragraphs below.

A1. A system for hydration monitoring and alerting, the system comprising: a server, wherein the server is configured to: obtain a set of photoplethysmogram (PPG) data samples for a set of population users; mark each PPG data sample of the set of PPG data samples with a selected label selected from a set of labels, to thereby obtain a set of training data, wherein the selected label indicates a hydration level of a population user of the set of population users at a specific time that corresponds to when a respective PPG data sample was obtained, and the set of labels includes at least two labels that indicate different hydration levels of the set of population users, and the at least two labels include a first label for dehydration and a second label for hydration; and generate a model based on the set of training data; and an interface-sensor system, wherein the user-sensor system includes: a PPG sensor configured to obtain a current PPG data sample for a user; and a user interface configured to obtain the current PPG data sample from the PPG sensor, and indicate a hydration state of the user based on the current PPG data sample, wherein the user interface is configured to: obtain the model; process the current PPG data sample through the model to obtain a hydration score for the user; based on the hydration score of the user, determine a hydration state of the user; and output an indicator based on the hydration state.

A2. The system of A1, wherein the set of labels includes at least five labels indicating different hydration levels.

A3. The system of A2, wherein the at least five labels correspond to 20% intervals on a hydration scale from 0 to 100%.

A4. The system of any of A1-A3, wherein to obtain the set of PPG data samples, the system is further configured to instruct the set of population users to perform a standardized routine to generate a distribution of different PPG data samples.

A5. The system of A4, wherein the standardized routine comprises performing incremental hydration over a plurality of hydration sessions.

A6. The system of A5, wherein for each hydration session, the system is configured to: determine a user has woken up from a threshold amount of sleep; obtain initial PPG data before hydration of the user; provide an instruction to drink an amount of water; determine the user has complied with the instruction; obtain incremental hydration PPG data for the hydration session; determine whether the incremental hydration PPG data for the hydration session satisfies a similarity condition with respect to a most recent incremental hydration PPG data for a most recent hydration session; and if so, determine to mark the initial PPG data with the first label.

A7. The system of A6, wherein the amount of water increases over the plurality of hydration sessions.

A8. The system of A6, wherein the hydration sessions are on successive days.

A9. The system of A6, wherein the hydration sessions are on days where the initial PPG data satisfies a baseline condition.

A10. The system of any of A1-A9, wherein the system is configured to mark samples with the first label based on an analysis of initial PPG data for a plurality of hydration sessions.

A11. The system of A10, wherein the first label indicates a least hydrated state of the user over the plurality of hydration sessions.

A12. The system of any of A1-A11, wherein the model is a machine learning model.

A13. The system of A12, wherein the machine learning model is a user-specific model.

A14. The system of A13, wherein the user-specific model is a calibrated version of a population model derived from the training data.

A15. The system of A12, wherein the machine learning model uses data associated with the user as a self-reference for determination of the hydration state.

A16. The system of A12, wherein the machine learning model is configured to receive input PPG data and determine an output hydration score based on the input PPG data.

A17. The system of A16, wherein the machine learning model is configured to operate on a plurality of parameters based on at least the first sample and the second sample.

A18. The system of A17, wherein the plurality of parameters includes statistics of PPG traces derived from the PPG data.

A19. The system of A17, wherein the plurality of parameters includes user demographic data.

A20. The system of A17, wherein the plurality of parameters includes user activity data and user sleep data.

A21. The system of any of A1-A20, wherein the PPG sensor is configured to obtain the current PPG data sample using green light with a wavelength between 525 nm and 540 nm.

A22. The system of any of A1-A21, wherein the user interface device is a wearable device.

A23. The system of A22, wherein the wearable device is a smartwatch.

A24. The system of any of A1-A23, wherein the user interface device is further configured to: determine that the hydration state indicates dehydration; and generate an alert to the user to increase fluid intake.

A25. The system of any of A1-A24, wherein the user interface device is further configured to: track the user's fluid intake over time; correlate the fluid intake with changes in the hydration state; and provide personalized hydration recommendations based on the correlation.

A26. The system of any of A1-A25, wherein the server is further configured to: receive updated PPG data samples from the user-sensor system over time; update the model based on the updated PPG data samples; and provide the updated model to the user interface device.

A27. The system of any of A1-A26, wherein the server is further configured to: aggregate anonymized hydration data from multiple users; and generate population-level hydration insights based on the aggregated data.

A28. The system of any of A1-A27, wherein the user interface device is further configured to: detect a sudden change in the hydration state; and trigger an emergency alert if the sudden change exceeds a predetermined threshold.

A29. The system of any of A1-A28, wherein the user interface device is further configured to: integrate the hydration state with other health metrics to provide a comprehensive health assessment.

A30. The system of A29, wherein the other health metrics include at least one of heart rate, blood pressure, body temperature, and physical activity level.

A31. The system of any of A1-A30, wherein the model comprises a data preprocessing block configured to perform denoising and short-time Fourier transform (STFT) on the current PPG data sample.

A32. The system of A31, wherein the model further comprises a first convolutional layer configured to apply first filters of a first predetermined size with ReLU activation.

A33. The system of A32, wherein the first convolutional layer is further configured to perform batch normalization and 3D max pooling on its output.

A34. The system of A32, wherein the model further comprises a second convolutional layer configured to apply second filters of a second predetermined size with ReLU activation.

A35. The system of A34, wherein the second convolutional layer is further configured to perform 3D max pooling on its output.

A36. The system of A34, wherein the model further comprises a third convolutional layer configured to apply third filters of a third predetermined size with padding and ReLU activation.

A37. The system of A36, wherein the third convolutional layer is further configured to perform 3D max pooling on its output.

A38. The system of A36, wherein the model further comprises a dense layer with a plurality of neurons and ReLU activation.

A39. The system of A38, wherein the model further comprises an output layer with at least one neuron and sigmoid activation.

A40. The system of A39, wherein the model further comprises a class prediction block configured to determine the hydration classification based on the output of the output layer.

A41. The system of any of A1-A40, wherein the model is configured to process the current PPG data sample through a series of convolutional layers, each applying an increasing number of filters.

A42. The system of A41, wherein the series of convolutional layers comprises at least three convolutional layers, with the number of filters changing from 12 to 24 to 48.

A43. The system of any of A1-A42, wherein the model is configured to use ReLU activation in all layers except for the output layer.

A44. The system of any of A1-A43, wherein the model is configured to use sigmoid activation in the output layer to compress the output to a range.

A45. The system of any of A1-A44, wherein the model is configured to perform 3D max pooling after each convolutional layer.

A46. The system of any of A1-A45, wherein the model is configured to flatten the output of the convolutional layers before passing it to a dense layer.

A47. The system of any of A1-A46, wherein the model is configured to use batch normalization after the first convolutional layer to accelerate and stabilize the training process.

A48. The system of any of A1-A47, wherein the model is configured to use padding in a third convolutional layer to preserve the spatial dimensions of the input feature maps.

A49. The system of any of A1-A48, wherein the model is configured to output a hydration score between 0 and 1, with values closer to 0 indicating dehydration and values closer to 1 indicating hydration.

A50. The system of any of A1-A49, wherein the model is configured to process the current PPG data sample through a neural network architecture comprising at least three convolutional layers, one dense layer, and one output layer.

B1. A system for hydration monitoring and alerting, the system comprising: a server, wherein the server is configured to: obtain a set of photoplethysmogram (PPG) data samples for a set of population users; mark each PPG data sample of the set of PPG data samples with a selected label selected from a set of labels, to thereby obtain a set of training data, wherein the selected label indicates a hydration level of a population user of the set of population users at a specific time that corresponds to when a respective PPG data sample was obtained, and the set of labels includes at least two labels that indicate different hydration levels of the set of population users, and the at least two labels include a first label for dehydration and a second label for hydration; and generate a model based on the set of training data; and an interface-sensor system, wherein the user-sensor system includes: a PPG sensor configured to obtain a current PPG data sample for a user; and a user interface configured to obtain the current PPG data sample from the PPG sensor, and indicate a hydration state of the user based on the current PPG data sample, wherein the user interface is configured to: obtain the model; process the current PPG data sample through the model to obtain a hydration score for the user; based on the hydration score of the user, determine a hydration state of the user; and output an indicator based on the hydration state.

B2. The system of B1, wherein the user interface is further configured to: determine that the user has woken up from a threshold sleep amount; obtain initial PPG data before the user hydrates as an initial maximum dehydration; monitor hydration levels using PPG data and the model; determine if there are deviations from a previous hydration state; and output alerts for detected deviations.

B3. The system of B2, wherein the user interface is further configured to: determine if a deviation represents a new maximum dehydration; and update a maximum dehydration value if the deviation represents a new maximum dehydration.

B4. The system of B3, wherein the user interface is further configured to: use the updated maximum dehydration value to adjust thresholds for generating alerts.

B5. The system of B3, wherein the user interface is further configured to: transmit the new maximum dehydration value to the server; and wherein the server is further configured to: update the model based on the new maximum dehydration value; and provide the updated model to the user interface for subsequent hydration state determinations.

B6. The system of any of B1-B5, wherein the user interface is further configured to: determine that the user has woken up from a threshold sleep amount; obtain initial PPG data before the user hydrates; provide instructions to drink a first amount of water; determine user compliance with the drinking instruction; obtain incremental hydration PPG data; check for plateau detection; and if plateau is not detected, determine a second amount and time frame for the user to consume water.

B7. The system of B6, wherein the user interface is further configured to: provide hydration session data to the server and/or the user if plateau is detected.

B8. The system of B7, wherein the server is further configured to: update the model based on the hydration session data, wherein the updated model improves inference capabilities for the specific user.

B9. The system of any of B1-B8, wherein the user interface is further configured to: determine a maximum dehydration state for the user; determine a maximum hydration state for the user; transmit the maximum dehydration state and the maximum hydration state to the server; and wherein the server is further configured to: update the model based on the maximum dehydration state and the maximum hydration state; and provide the updated model to the user interface, wherein the updated model may improve inference capabilities for determining the hydration state of the user.

C1. A system for hydration monitoring and alerting, the system comprising: a server, wherein the server is configured to: obtain a set of photoplethysmogram (PPG) data samples for a set of population users; mark each PPG data sample of the set of PPG data samples with a selected label selected from a set of labels, to thereby obtain a set of training data, wherein the selected label indicates a hydration level of a population user of the set of population users at a specific time that corresponds to when a respective PPG data sample was obtained, and the set of labels includes at least two labels that indicate different hydration levels of the set of population users, and the at least two labels include a first label for dehydration and a second label for hydration; and generate a model based on the set of training data; and an interface-sensor system, wherein the user-sensor system includes: a PPG sensor configured to obtain a current PPG data sample for a user; and a user interface configured to obtain the current PPG data sample from the PPG sensor, and indicate a hydration state of the user based on the current PPG data sample, wherein the user interface is configured to: obtain the model; process the current PPG data sample through the model to obtain a hydration score for the user; based on the hydration score of the user, determine a hydration state of the user; and output an indicator based on the hydration state.

C2. The system of C1, wherein the interface-sensor system further comprises a temperature sensor configured to obtain body temperature measurements, and wherein the user device is further configured to process the body temperature measurements as additional input parameters alongside the current PPG data sample through the model.

C3. The system of C2, wherein the temperature sensor is configured to obtain body temperature measurements continuously or at regular intervals corresponding to PPG data collection, enabling correlation of temperature trends with PPG signal changes.

C4. The system of C2, wherein the model is configured to learn complex relationships between temperature variations and PPG signal characteristics to identify dehydration states more accurately than PPG data alone.

C5. The system of any of C1-C4, wherein the server and the user interface are configured to apply a bandpass filter to the PPG data samples to extract frequency components within a range of 0.5 to 6 Hz before generating the model or processing the current PPG data sample through the model to obtain the hydration score for the user.

C6. The system of C5, wherein the bandpass filter eliminates low-frequency drift artifacts below 0.5 Hz and high-frequency noise above 6 Hz that do not contribute meaningful physiological information for hydration assessment.

C7. The system of any of C1-C6, wherein the model operates on a reduced set of feature parameters comprising 112 parameters selected from an initial set of 512 parameters to improve computational efficiency while maintaining prediction accuracy.

C8. The system of any of C1-C7, wherein the user interface is configured to implement a non-linear hydration scale for training data collection.

C9. The system of C8, wherein the non-linear hydration scale includes fluid volume increments of 0 ml, 500 ml, 700 ml, 800 ml, 900 ml, and 1000 ml.

C10. The system of C8, wherein the non-linear hydration scale provides finer resolution at higher dehydration levels.

C11. The system of C8, wherein the non-linear hydration scale provides 100 ml increments between 700 ml and 1000 ml to reflect higher probability of moderate fluid loss.

C12. The system of C8, wherein for individual-based training targeting athletes, the hydration scale extends beyond 1000 ml with additional increments of 200-250 ml, potentially reaching 1.5 liters or higher.

C13. The system of any of C1-C12, wherein the server or the user interface are configured to correlate fluid intake requirements with Body Mass Index (BMI) of users.

C14. The system of C13, wherein the BMI correlation is based on blood volume scaling with body mass, with circulating blood volumes ranging from approximately 4.9 liters in normal-weight individuals to 7 liters in individuals with elevated BMI.

C15. The system of C13, wherein the server or the user interface adjusts fluid intake recommendations during training data collection or adjusts fluid intake during routines based on user BMI to ensure data accurately reflects the relationship between PPG signals and hydration states across different body compositions.

C16. The system of any of C1-C15, wherein the server is configured to implement a two-tier training approach comprising: a population-based model trained on PPG data from multiple subjects across diverse demographic and physiological characteristics; and an individual-based model created through user-specific training protocols for enhanced accuracy.

C17. The system of C16, wherein new users begin with the population-based model for immediate hydration monitoring capability and optionally upgrade to individual-based training for enhanced precision.

C18. The system of C16, wherein the individual-based model involves the user performing a complete hydration protocol personally, generating PPG data specific to their physiological responses including cardiovascular response variations, skin properties, and personal hydration kinetics.

C19. The system of C16, wherein the individual training process extends beyond a standard 1-liter population scale and continues until plateau detection indicates the user's personal maximum hydration state has been achieved.

C20. The system of any of C1-C19, wherein the model comprises: a preprocessing layer configured to apply bandpass filtering and feature parameter reduction; convolutional layers configured to extract spatial features from preprocessed PPG data; and dense layers configured to process temperature data as additional input parameters alongside PPG-derived features.

C21. The system of any of C1-C20, wherein the system is configured to detect plateau states during individual training by analyzing PPG data using statistical measures including moving average stabilization, standard deviation reduction, and slope analysis approaching zero.

C22. The system of C21, wherein plateau detection involves comparing current PPG signals to previous hydration session data using correlation coefficients, relative change analysis, and statistical distance measures to determine when maximum hydration has been reached.

Other aspects of the disclosure will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A system for hydration monitoring and alerting, the system comprising:
a server, wherein the server is configured to:
obtain a set of photoplethysmogram (PPG) data samples for a set of population users;
mark each PPG data sample of the set of PPG data samples with a selected label selected from a set of labels, to thereby obtain a set of training data,
wherein the selected label indicates a hydration level of a population user of the set of population users at a specific time that corresponds to when a respective PPG data sample was obtained, and
the set of labels includes at least two labels that indicate different hydration levels of the set of population users, and
the at least two labels include a first label for dehydration and a second label for hydration; and
generate a model based on the set of training data; and
an interface-sensor system, wherein the user-sensor system includes:
a PPG sensor configured to obtain a current PPG data sample for a user; and
a user interface configured to obtain the current PPG data sample from the PPG sensor, and indicate a hydration state of the user based on the current PPG data sample, wherein the user interface is configured to:
obtain the model;
process the current PPG data sample through the model to obtain a hydration score for the user;
based on the hydration score of the user, determine a hydration state of the user; and
output an indicator based on the hydration state.

2. The system of claim 1, wherein the interface-sensor system further comprises a temperature sensor configured to obtain body temperature measurements, and wherein the user interface is further configured to process the body temperature measurements as additional input parameters alongside the current PPG data sample through the model.

3. The system of claim 2, wherein the temperature sensor is configured to obtain body temperature measurements continuously or at regular intervals corresponding to PPG data collection, enabling correlation of temperature trends with PPG signal changes.

4. The system of claim 2, wherein the model is configured to learn complex relationships between temperature variations and PPG signal characteristics to identify dehydration states more accurately than PPG data alone.

5. The system of claim 1, wherein the server and the user interface are configured to apply a bandpass filter to the PPG data samples to extract frequency components within a range of 0.5 to 6 Hz before generating the model or processing the current PPG data sample through the model to obtain the hydration score for the user.

6. The system of claim 5, wherein the bandpass filter eliminates low-frequency drift artifacts below 0.5 Hz and high-frequency noise above 6 Hz that do not contribute meaningful physiological information for hydration assessment.

7. The system of claim 1, wherein the user interface is configured to implement a non-linear hydration scale for training data collection.

8. The system of claim 1, wherein the user interface is further configured to: determine that the user has woken up from a threshold sleep amount; obtain initial PPG data before the user hydrates as an initial maximum dehydration; monitor hydration levels using PPG data and the model; determine if there are deviations from a previous hydration state; and output alerts for detected deviations.

9. The system of claim 8, wherein the user interface is further configured to: determine if a deviation represents a new maximum dehydration; and update a maximum dehydration value if the deviation represents a new maximum dehydration.

10. The system of claim 9, wherein the user interface is further configured to: transmit the new maximum dehydration value to the server; and wherein the server is further configured to: update the model based on the new maximum dehydration value; and provide the updated model to the user interface for subsequent hydration state determinations.

11. The system of claim 1, wherein the user interface is further configured to: determine that the user has woken up from a threshold sleep amount; obtain initial PPG data before the user hydrates; provide instructions to drink a first amount of water; determine user compliance with the drinking instruction; obtain incremental hydration PPG data; check for plateau detection; and if plateau is not detected, determine a second amount and time frame for the user to consume water.

12. The system of claim 11, wherein the user interface is further configured to: provide hydration session data to the server and/or the user if plateau is detected.

13. The system of claim 1, wherein the set of labels includes at least five labels indicating different hydration levels.

14. The system of claim 13, wherein the at least five labels correspond to 20% intervals on a hydration scale from 0 to 100%.

15. The system of claim 1, wherein to obtain the set of PPG data samples, the system is further configured to instruct the set of population users to perform a standardized routine to generate a distribution of different PPG data samples.

16. The system of claim 15, wherein the standardized routine comprises performing incremental hydration over a plurality of hydration sessions, and wherein for each hydration session, the system is configured to: determine a user has woken up from a threshold amount of sleep; obtain initial PPG data before hydration of the user; provide an instruction to drink an amount of water; determine the user has complied with the instruction; obtain incremental hydration PPG data for the hydration session; determine whether the incremental hydration PPG data for the hydration session satisfies a similarity condition with respect to a most recent incremental hydration PPG data for a most recent hydration session; and if so, determine to mark the initial PPG data with the first label.

17. The system of claim 16, wherein the amount of water increases over the plurality of hydration sessions.

18. The system of claim 1, wherein the model is configured to process the current PPG data sample through a series of convolutional layers, each applying an increasing number of filters.

19. The system of claim 1, wherein the model is configured to output a hydration score between 0 and 1, with values closer to 0 indicating dehydration and values closer to 1 indicating hydration.

20. The system of claim 1, wherein the model is configured to process the current PPG data sample through a neural network architecture comprising at least three convolutional layers, one dense layer, and one output layer.
